SEQUENCE LISTING (35 sequences)

SEQ ID NO: 1 - protein, 7 aa, Monkey virus
PKKKRKV

SEQ ID NO: 2 - protein, 6 aa, Homo sapiens
ARRRRP

SEQ ID NO: 3 - protein, 10 aa, Unknown Organism
  Description of Unknown Organism NF-kappa-B p50 targeting sequence
EEVQRKRQKL

SEQ ID NO: 4 - protein, 9 aa, Unknown Organism
  Description of Unknown Organism NF-kappa-B p65 targeting sequence
EEKRKRTYE

SEQ ID NO: 5 - protein, 20 aa, Xenopus sp.
AVKRPAATKKAGQAKKKKLD

SEQ ID NO: 6 - protein, 31 aa, Homo sapiens
MASPLTRFLSLNLLLLGESILGSGEAKPQAP

SEQ ID NO: 7 - protein, 21 aa, Homo sapiens
MSSFGYRTLTVALFTLICCPG

SEQ ID NO: 8 - protein, 51 aa, Homo sapiens
PQRPEDCRPRGSVKGTGLDFACDIYIWAPLAGICVALLLSLIITLICYHSR

SEQ ID NO: 9 - protein, 33 aa, Homo sapiens
MVIIVTVVSVLLSLFVTSVLLCFIFGQHLRQQR

SEQ ID NO: 10 - protein, 37 aa, Unknown Organism
  Description of Unknown Organism DAF targeting sequence
PNKGSGTTSGTTRLLSGHTCFTLTGLLGTLVTMGLLT

SEQ ID NO: 11 - protein, 14 aa, Unknown Organism
  Description of Unknown Organism C-src targeting sequence
MGSSKSKPKDPSQR

SEQ ID NO: 12 - protein, 26 aa, Unknown Organism
  Description of Unknown Organism GRK6 targeting sequence
LLQRLFSRQDCCGNCSDSEEELPTRL

SEQ ID NO: 13 - protein, 20 aa, Mus musculus
KQFRNCMLTSLCCGKNPLGD

SEQ ID NO: 14 - protein, 19 aa, Homo sapiens
LNPPDESGPGCMSCKCVLS

SEQ ID NO: 15 - protein, 15 aa, Unknown Organism
  Description of Unknown Organism rab-5A targeting sequence
LTEPTQPTRNQCCSN

SEQ ID NO: 16 - protein, 5 aa, Unknown Organism
  Description of Unknown Organism Lamp-2 targeting sequence
KFERQ

SEQ ID NO: 17 - protein, 36 aa, Unknown Organism
  Description of Unknown Organism Lamp-1 targeting sequence
MLIPIAGFFALAGLVLIVLIAYLIGRKRSHAGYQTI

SEQ ID NO: 18 - protein, 35 aa, Unknown Organism
  Description of Unknown Organism Lamp-2 targeting sequence
LVPIAVGAALAGVLILVLLAYFIGLKHHHAGYEQF

SEQ ID NO: 19 - protein, 27 aa, Saccharomyces sp.
MLRTSSLFTRRVQPSLFSRNILRLQST

SEQ ID NO: 20 - protein, 25 aa, Saccharomyces sp.
MLSLRQSIRFFKPATRTLCSSRYLL

SEQ ID NO: 21 - protein, 64 aa, Saccharomyces sp.
MFSMLSKRWAQRTLSKSFYSTATGAASKSGKLTQKLVTAGVAAAGITASTLLYADSLTAEAMTA

SEQ ID NO: 22 - protein, 41 aa, Saccharomyces sp.
MKSFITRNKTAILATVAATGTAIGAYYYYNQLQQQQQRGKK

SEQ ID NO: 23 - protein, 4 aa, Unknown Organism
  Description of Unknown Organism Calreticulin targeting sequence
KDEL

SEQ ID NO: 24 - protein, 15 aa, Homo sapiens
LYLSRRSFIDEKKMP

SEQ ID NO: 25 - protein, 9 aa, Unknown Organism
  Description of Unknown Organism Cyclin B1 destruction sequence
RTALGDIGN

SEQ ID NO: 26 - protein, 5 aa, Unknown Organism
  Description of Unknown Organism Lamp-2 destruction sequence
KFERQ

SEQ ID NO: 27 - protein, 4 aa, Unknown Organism
  Description of Unknown Organism Consensus sequence
PEST

SEQ ID NO: 28 - protein, 6 aa, Artificial Sequence
  Description of Artificial Sequence Synthetic 6X His tag
HHHHHH

SEQ ID NO: 29 - protein, 20 aa, Artificial Sequence
  Description of Artificial Sequence Synthetic Gly-Ser linker
GGGGSGGGGSGGGGSGGGGS

SEQ ID NO: 30 - protein, 35 aa, Unknown Organism
  Description of Unknown Organism Dimerization domain sequence
IQRMKQLEDKVEELLSKNYHLENEVARLKKLVGER

SEQ ID NO: 31 - protein, 6 aa, Unknown Organism
  Description of Unknown Organism Dimerization domain sequence
SKVILF

SEQ ID NO: 32 - protein, 22 aa, Unknown Organism
  Description of Unknown Organism Leucine zipper domain sequence
LXXXXXXLXXXXXXLXXXXXXL

SEQ ID NO: 33 - protein, 73 aa, Unknown Organism
  Description of Unknown Organism bHLH dimerization sequence
DENSTAPKRLIVMAGSNTFYWCPHKRLIVMTLIVMXXSTAVLIVMSTACKRXVMFYHLIVMTAPPLIVMRKHQ

SEQ ID NO: 34 - protein, 943 aa, Homo sapiens
MAASGLDHLKNGYRRRFCRPSRARDINTEQGQNVLEILQDCFEEKSLANDFSTNSTKSVPNSTRKIKDTCIQSPSKECQKSHPKSVPVSSKKKEASLQFVVEPSEATNRSVQAHEVHQKILATDVSSKNTPDSKKISSRNINDHHSEADEEFYLSVGSPSVLLDAKTSVSQNVIPSSAKKRETYTFENSVNMLPSSTEVSVKTKKRLNFDDKVMLKKIEIDNKVSDEEDKTSEGQERKPSGSSQNRIRDSEYEIQRQAKKSFSTLFLETVKRKSESSPIVRHAATAPPHSCPPDDTKLIEDEFIIDESDQSFASRSWITIPRKAGSLKQRTISPAESTALFQGRKSREKHHNILPKTLANDKHSHKPHPVETSQPSDKTVLDTSYALIDETVNNYRSTKYEMYSKNAEKPSRSKRTIKQKQRRKFMAKPAEEQLDVGQSKDENIHTSHITQDEFQRNSDRNMEEHEEMGNDCVSKKQMPPVGSKKSSTRKDKEESKKKRFSSESKNKLVPEEVTSTVTKSRRISRRPSDWWVVKSEESPVYSNSSVRNELPMHHNSSRKSTKKTNQSSKNIRKKTIPLKRQKTATKGNQRVQKFLNAEGSGGIVGHDEISRCSLSEPLESDEADLAKKKNLDCSRSTRSSKNEDNIMTAQNVPLKPQTSGYTCNIPTESNLDSGEHKTSVLEESGPSRLNNNYLMSGKNDVDDEEVHGSSDDSKQSKVIPKNRIHHKLVLPSNTPNVRRTKRTRLKPLEYWRGERIDYQGRPSGGFVISGVLSPDTISSKRKAKENIGKVNKKSNKKRICLDNDERKTNLMVNLGIPLGDPLQPTRVKDPETREIILMDLVRPQDTYQFFVKHGELKVYKTLDTPFFSTGKLILGPQEEKGKQHVGQDILVFYVNFGDLLCTLHETPYILSTGDSFYVPSGNYYNIKNLRNEESVLLFTQIKR

SEQ ID NO: 35 - DNA, 3132 bp, Homo sapiens
cggatcgcagctctcgcggcagtcgcctgagacttaaggttattgcttggccgcggcctggtattccggcgattcgtttcttgctcggcttcctggagctgtggtccgtgtgggcttccacctcagacagttgcgctggctcagcggggccggaacatggctgcgtccggtctggatcatctcaaaaatggctacagaagaagattttgtcgaccttccagggcacgtgacattaacacagagcaaggccagaatgttctggaaatcttacaagactgttttgaagaaaaaagtcttgccaatgattttagtacaaattctacaaaatcagtgcctaattcaacacgcaaaataaaagacacttgtattcagtcaccaagcaaagagtgccagaaatcacatccaaagtcagttccagtttcttcaaagaagaaagaagcctctctacagtttgttgtagaaccaagtgaagccacaaacagatcagttcaggcccatgaagttcatcagaaaattctggcaactgatgttagttccaaaaatacacctgactcgaaaaaaatatcaagtagaaacataaatgatcatcacagtgaagctgatgaagaattttacttatccgttggctcaccttctgttcttttggatgcaaaaacatctgtatcacaaaatgttattccatctagtgccaaaaagagagagacttacacttttgaaaattcagtaaatatgctgccttcaagtacagaggtttcagttaaaaccaaaaaaaggttaaactttgatgataaagttatgttaaagaaaatagaaatagataataaagtatcagatgaagaggataaaacatcggaaggacaagaaagaaaaccatcaggatcatctcagaatagaatacgagattcagaatatgaaattcaacgacaagctaaaaaaagtttttcaacattgtttttagaaacagtaaaacgaaaaagtgaatccagtcccattgttaggcatgcggcaactgctccacctcattcgtgtcctcccgatgatacgaagttgatagaggatgaatttataattgatgagtcggatcaaagttttgccagtagatcttggattacaataccaagaaaggcagggtctctgaaacaacgcacaatatccccggctgagagcactgcactctttcaaggtagaaagtcaagagaaaagcatcataatatattacctaagactttggcaaatgacaaacattcccataaacctcacccagtagagacatctcagccctctgataaaacagtactggatacaagttatgctttgatagatgaaacagtaaataattatagatctacaaaatatgaaatgtattccaagaatgcagaaaaaccatctagaagcaaaaggactataaaacaaaaacagagaagaaaattcatggctaaaccagctgaagaacagcttgatgtgggacagtctaaagatgaaaacatacatacatcacatattacccaagacgaatttcaaagaaattcagacagaaatatggaagagcatgaagagatgggaaatgattgtgtttccaaaaaacagatgccacctgtgggaagcaagaaaagtagcactagaaaagataaggaagaatctaaaaagaagcgcttttccagtgagtccaagaacaaacttgtacctgaagaagtgacttcaactgtcacgaaaagtcgaagaatttccaggcgtccatctgattggtgggtggtaaaatcagaggagagtcctgtttatagcaattcttcagtaagaaatgaattaccaatgcatcacaatagtagccgaaaatctactaagaaaacaaatcagtcatctaagaatattaggaaaaaaactattccacttaaaaggcagaagacagcaactaaaggcaaccaaagagtacagaagtttttaaatgctgaaggttctggaggtatcgttggtcatgatgaaatttccagatgttcactgagtgagccattggaaagtgatgaggcagacttggctaagaagaaaaatcttgattgttctagatctacaagaagctcaaagaatgaagataacattatgactgcacagaatgttcccctaaagcctcagaccagtggatatacatgtaatataccaacagagtcaaacttggattctggagagcataagacttcagttttagaggaaagtggaccttccaggctcaataataattatttaatgtctggaaagaatgatgtggatgatgaggaagttcatggaagttcagatgactcaaaacaatctaaagtgataccaaagaacagaatccatcacaaactagtattgccctccaacacaccaaatgttcgcaggaccaagagaacacgtttgaaacctttggagtactggcgaggagagcgaatagattatcaaggaaggccatcaggaggattcgtgattagtggagtactatctccagacacaatatcgtctaaaaggaaggcaaaagaaaatattggaaaagtcaacaaaaaatctaataagaaaaggatctgtcttgataacgatgaaagaaagactaacttaatggtaaatctaggtatacctcttggagatcctttgcagccaacgagggtaaaggacccagaaacaagagagattattctcatggatcttgtaaggccacaagatacatatcaattttttgttaagcatggtgagttgaaggtatacaagacattggatacaccctttttttctactgggaaattgatattaggaccacaagaagaaaagggaaagcagcatgttggccaggatatattggttttttatgttaactttggtgaccttttgtgtactttacatgaaacaccttatatattaagtactggggattcgttctatgttccttcaggtaactattataacatcaaaaatctccggaatgaggaaagtgttcttctttttactcagataaaaagatgaaagatcaaccaaccttaaatatatgtatgtatatatgtatatgtaaaaacagtttgtatagttggaatatttgtctttgtaattacttgtgatgttttaaaataaaaattttattcagttttgtgtaaaaaaaaaaaaaaaaaa

(19) United States
(12) Patent Application Publication
(10) Pub. No.: US 2005/0277116 A1
(43) Pub. Date: Dec. 15, 2005

(54) Compositions and methods for the identification of protein interactions in vertebrate cells

(76) Inventors: Frank McKeon, Boston, MA (US); Annie Yang, Boston, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/475,204

(22) PCT Filed: Apr. 22, 2002
(86) PCT No.: PCT/US02/13008

§ 371 Date: Apr. 30, 2004

Related U.S. Application Data
(60) Provisional application No. 60/285,509, filed on Apr. 20, 2001.

(51) Int. Cl. C12Q 1/68; G01N 33/53; G01N 33/567; C12N 15/85

(52) U.S. Cl. 435/6; 435/7.2; 435/455

(57) ABSTRACT

The present invention relates to a two-hybrid system for studying protein-protein interactions in mammalian host cells. The invention provides methods, reagents and kits for carrying out the two-hybrid screen. Accordingly the invention provides a bait construct that is capable of targeting a bait protein to a specific subcellular locale within the host cell. The invention also provides a prey construct that contains a detection sequence fused to a prey protein. The bait and prey constructs are introduced into a host cell under conditions that promote expression of the bait and prey constructs. A positive bait/prey interaction can be detected by comparing the subcellular localization of the prey construct in relation to the bait construct. The invention also provides methods for screening for compounds capable of disrupting protein-protein interaction and methods for detecting interactions between proteins and small molecule compounds.

### BACKGROUND OF THE INVENTION

Protein-protein interactions are of paramount and fundamental interest in biological systems. These interactions are involved in a wide variety of important biological reactions, including the assembly of enzyme subunits, in antigen-antibody reactions, in supramolecular structures of ribosomes, filaments, and viruses, in recognition and transport, in transcription regulation, and in ligand-receptor interactions. In addition, the area of protein-protein interactions has received significant attention in the area of signal transduction and biochemical pathway analysis.

Traditionally, protein-protein interactions were evaluated using biochemical techniques, including chemical cross-linking, co-immunoprecipitation and co-fractionation and -purification. Recently, genetic systems have been described to detect protein-protein interactions. The first work was done in yeast systems, and was termed the “yeast two-hybrid” system. The basic system requires a protein-protein interaction in order to turn on transcription of a reporter gene. Similar systems operating in bacteria and mammalian cells have also been developed. See Fields et al., Nature 340:245 (1989); Vasavada et al., PNAS USA 88:10686 (1991); Fearon et al., PNAS USA 89:7958 (1992); Dang et al., Mol. Cell. Biol. 11:954 (1991); Chien et al., PNAS USA 88:9578 (1991); and U.S. Pat. Nos. 5,283,173, 5,667,973, 5,468,614, 5,525,490, and 5,637,463. (need patent for bacterial two hybrid if not here)

However, while the yeast system works well, it is unsuitable for use in mammalian systems for a variety of reasons. Furthermore, the existing mammalian two-hybrid systems are neither suitable for a wide variety of cells, nor flexible, as they generally require quite highly specialized conditions. Finally, these systems tend to have high background signals from non-specific interactions, giving rise to unacceptable levels of “false positives”.

A number of factors make a mammalian two-hybrid system highly desirable. First of all, post-translational modifications of proteins may contribute significantly to their ability to interact, and yet such modifications may not be supported in a yeast environment. Consequently, proteins that would interact with correct post-translational processing may not be identified in a yeast system. Certain post-translational modifications that influence protein-protein interactions only occur secondarily to activation of particular signaling cascades. As yeast lacks many of the cell surface receptors that trigger such cascades, discovery of signal-dependent modifications and protein-protein interactions is generally not feasible in a yeast cell. However, several decades of research have been devoted to initiating such signaling cascades in mammalian cells.

A mammalian two-hybrid system operable in a variety of mammalian cell types would be highly desirable, since the regulation, induction, processing, etc. of specific proteins within a particular cell type can vary significantly, it would thus be a distinct advantage to assay for relevant protein-protein interactions in the relevant cell type. For example, proteins involved in a disease state could be tested in the relevant disease cells, resulting in a higher chance of identifying important protein interactions. Similarly, for testing of random proteins, assaying them under the relevant cellular conditions will give the highest chance of positive results. Furthermore, the mammalian cells can be tested under a variety of experimental conditions that may affect intracellular protein-protein interactions, such as in the presence of hormones, drugs, growth factors and cytokines, cellular and chemical stimuli, etc., that may contribute to conditions which can effect protein-protein interactions.

Thus, a robust and adaptable mammalian two-hybrid system that can work in a wide variety of mammalian cell types is highly desirable.

In certain embodiments, the two hybrid assay systems of the prior art have been modified to detect ligand-dependent interaction of proteins. In these “three hybrid assay” systems, the ability of another protein or small organic molecule to mediate interaction of two proteins is detected.

Accordingly, it is an object of the invention to provide compositions and methods useful in two-hybrid assay systems that can be utilized reproducibly and stably in vertebrate cells, especially mammalian cells.

Another object of the invention to provide compositions and methods useful in a three-hybrid systems that can be utilized reproducibly and stably in vertebrate cells, especially mammalian cells.

### SUMMARY OF THE INVENTION

The present invention relates to, inter alia, compositions and methods for identification of protein-protein interactions and protein-compound interactions in mammalian cells.

In one aspect, the invention provides a method for detecting protein interactions in a host cell, comprising:
(i) providing a host cell including:
   (a) a nucleic acid encoding a bait fusion protein comprising a bait polypeptide sequence fused to a targeting domain that targets the bait fusion protein to a specific subcellular location within the host cell;
   (b) a nucleic acid sequence encoding a prey fusion protein comprising a prey polypeptide sequence fused to one or more detection domains that can be detected in a transcriptionally-independent manner in the host cell to determine if the prey fusion protein is associated in a complex with the bait fusion protein or not;
(ii) detecting the subcellular location of the prey protein within the host cell; wherein accumulation of the prey fusion at the same subcellular pattern as the bait fusion indicates that the prey fusion protein is associated in a complex with the bait fusion protein.
   The subject method can be used to identify or measure direct or indirect protein-protein interactions in protein complexes, as well as identify or measure ligand-mediated protein-protein interactions. In either case, the assay can be derived to permit observation of the effects post-translational modifications, such as phosphorylation or the like, may have on the formation of protein complexes. In the case of ligand-mediated complexes, the assay can be used to identify or measure the ability of a ligand (such as a protein, peptide or small organic molecule) to directly bind both the bait and prey fusion proteins. It can also be used to identify or measure the ability of a ligand to act as an allosteric agent, e.g., binding one of the bait or prey fusion proteins and inducing a conformation change in the protein which effects the formation of bait-prey complexes.

In still other embodiments, the subject method and system can be used to identify agents which can disrupt the formation of prey-bait complexes, e.g., either directly by competitive binding to the bait or prey polypeptide sequences, indirectly by allosterically modifying one of the bait or prey polypeptide sequences, or by inhibiting post-translational modification of one or both of the bait and prey polypeptide sequences.

In one embodiment, the targeting domain is capable of targeting the bait fusion to an intracellular structure or organelle selected from the group consisting of the nucleus, nucleoli, telomeres, kinetochores, nuclear envelope, chromosomes, chromatin, cytoplasm, endoplasmic reticulum, Golgi, centrosome, transgolgi network, cytoplasmic vesicles, mitochondria, secretory vesicles, lysosome, plasma membrane, intracellular membrane vesicles, nuclear membranes, synapses or basolatemal membranes. Preferably the targeting domain causes localization of the prey fusion protein, should the bait and prey proteins form a complex, in a manner in which localization of the prey fusion protein can be discerned by microscopy or flow cytometry.

In certain embodiments, the intracellular localization of the prey fusion protein is determined by direct visualization (including by automated processes) of the localization pattern of the prey fusion proteins, as opposed to a transcriptionally-dependent readout such as expression of a reporter gene. In preferred embodiments, the localization of the prey fusion proteins can be determined within minutes of expression of the bait and prey proteins in the host cells, e.g., preferably less 120 minutes, even more preferably less than 60, 45, 30, 15 or even 10 minutes.

In certain preferred embodiments, the targeting domain is capable of targeting the bait fusion protein to the kinetochores. Preferred domains for targeting to the kinetochore are those derived from a CENP-A, CENP-B, CENP-C, CENP-E, CENP-F, Bub1, Bub3, MAD3L or MAD2 protein, or a portion thereof capable of targeting the bait fusion to the kinetochores. In a particularly preferred embodiment, the targeting domain comprises at least amino acids 373-943 of the human CENP-C sequence or a homolog thereof which retains the ability to be docked to the kinetochore. In certain preferred embodiments, the targeting domain associates with the kinetochore structure with a dissociation constant (k_{d}) of 1 mM or less, and even more preferably with a k_{d} less than 10 μM, 1 μM, 100 nM, 10 n, or even 1 nM.

In other preferred embodiments, the targeting domain is capable of targeting the bait fusion to the nuclear envelope. Preferred domains for targeting to the nuclear envelope are those derived from a lamin A, lamin B, lamin C, emerins or porins protein, or a portion thereof capable of targeting the bait fusion to the nuclear envelope.

In certain embodiments, the detection domain fused to the prey protein is a fluorescent protein or a luminescent protein. Suitable detection domains include, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine, red fluorescent protein from discosoma (dsRED), and variants thereof. Preferred detection sequences are the green fluorescent protein and the S26T/V 163A mutant of the green fluorescent protein.

In certain embodiments, the bait and/or prey fusions include an instability sequence. Preferred instability sequences give the resulting fusion protein a shorter intracellular half-life (e.g., at least 50 percent shorter, even more preferably at least 75, 90, 95 or even 99 percent shorter) when not sequestered at the intracellular site to which the bait fusion protein is directed relative to when it is. In certain embodiments, the instability sequence includes a CENP-C instability domain, e.g., the fusion protein includes at least amino acids 249-323 of the human CENP-C sequence or a sequence homologous thereto and causes degradation of the protein if not docked at the kinetochore. In certain preferred embodiments, the prey fusion protein includes the instability sequence and the half life of the detection domain is shortened if the prey fusion protein is not localized with the bait fusion protein.

In certain embodiments, the bait and/or prey fusions may also include one or more amino acid sequences selected from the group consisting of rescue sequences and/or oligomerization domains (such as a dimerization domain). Preferred rescue sequences are the His₆ tag, myc tag, flu tag, lacZ, GST, Strep tag I and Strep tag II. Preferred oligomerization domains comprise the dimerization domain from the yeast GCN4 protein or from p53.

In certain embodiments, the nucleic acid sequences encoding the bait and prey fusions are each contained on a vector, which may be the same vector or separate vectors. Exemplary vectors include integrative as well as episomal vectors. In certain preferred embodiments, the vector is a retroviral vector. Preferred plasmids of the invention include pCEP4, pCI-NEO, pBI-EGF, pcDNAI/amp, pcDNAI/neo, pRc/CMV, pSV2gpt, pSV2neo, pSV2-dhfr, pTk2, pRSVneo, pMSG, pSVT7, pko-neo and pHyg. The bait and prey fusions may be contained on the same or separate vectors and may be operably linked to the same or different transcriptional regulatory sequences.

In certain embodiments, the the vector includes a recovery element, e.g., a nucleic acid sequence which binds to an agent which permits affinity purification of the vector from a cell lysate. An exemplary recovery element is a lacO sequence which binds to a lacZ protein.

In certain embodiments, the vectors encoding the bait and/or prey fusions may also include one or more selection sequence. Preferred selection sequences are neomycin, blastocidin, bleomycin, puromycin and hygromycin, and the bait and prey fusions may contain the same or different selection sequences.

In certain embodiments, the nucleic acid sequences encoding the bait and prey fusions further comprise a promoter sequence. The promoter may constitutive or inducible. Preferred promoters include the CMV, SV40, SRα, RSV, TK and beta-globin promoters.

Preferred host cells of the invention include mammalian cells, such as primate, murine, porcine, rat cells and the like. Primate cells, particularly human cells are expecially preferred in certain embodiments. However, in the use of embodiments in which the bait fusion protein localizes to the kinetochore, any cell which includes chromosomal structures including kinetochores can be used.

In certain embodiments, libraries of bait and/or prey fusion proteins (and the corresponding coding sequences) can be constructed by fusing a variegated library of coding sequences to a common targeting sequence (to form a library of bait proteins) or to a common detection sequence (to form a library of prey proteins).

Identification of a protein complexes including the bait and prey fusion proteins may be carried out by screening a single bait fusion against a single prey fusion, by screening a single bait fusion against a library of prey fusions to identify prey fusions which are capable of interacting with the bait fusion, by screening a single prey fusion against a library of bait fusions to identify bait fusions which are capable of interacting with the prey fusion, or by screening a library of prey fusions against a library of bait fusions in order to identify bait and prey fusions capable of interacting.

In a preferred embodiment, the method further comprises the step of determining the specific subcellular localization of the prey fusion within the host cell. Preferably, the expression of the prey fusion is detected using FACS analysis.

In another aspect, the invention provides a method for detecting a protein-compound interaction in a mammalian cell, comprising:
(i) constructing a nucleic acid encoding a bait fusion comprising a sequence capable of targeting the bait fusion to a specific subcellular location within a cell fused in frame with receptor protein;
(ii) constructing a compound comprising a ligand for the receptor of the bait fusion fused to a compound;
(iii) constructing a nucleic acid sequence encoding a prey fusion comprising a detection sequence capable of permitting determination of the subcellular localization of the prey fusion within a cell fused in frame with a prey protein;
(iv) introducing the nucleic acids encoding the bait and prey fusions and the receptor-compound fusion into a host cell;
(v) detecting the subcellular location of the prey protein within the host cell; wherein the ligand-compound fusion is localized to the same subcellular location to which the bait fusion was targeted via interaction of the ligand with the receptor portion of the bait fusion; and
wherein accumulation of the prey fusion at the same subcellular location to which the bait fusion was targeted is indicative of an interaction between the compound and the prey fusion.

In a preferred embodiment, a library of compounds is fused to the ligand and is screened against a particular prey fusion to find compounds which interact with the prey fusion. In another preferred embodiment, a single compound is fused to the ligand and is screened against a library of prey fusions to find a prey fusion which interacts with the compound. In a particularly preferred embodiment, the bait fusion comprises the ecdysone receptor, or a portion thereof which is capable of binding to the ecdysone ligand, and the ligand is ecdysone.

In another aspect, the invention provides a method for screening for compounds which inhibit or potentiate a protein-protein interaction in a mammalian cell, comprising:
(i) constructing a nucleic acid encoding a bait fusion comprising a subcellular localization domain and a bait protein;
(ii) constructing a nucleic acid encoding a prey fusion comprising a reporter sequence and a prey protein;
(iii) introducing the nucleic acids encoding the bait and prey fusions into a host cell;
(iv) contacting the cell expressing the bait and prey fusions with a test compound;
(iv) detecting the subcellular location of the prey protein within the host cell in the presence and absence of the test compound;
wherein a change in the accumulation of the prey fusion at the same subcellular location to which the bait fusion was targeted in the presence of the test compound as compared to the accumulation in the absence of the test compound is indicative of a test compound capable of inhibiting or potentiating an interaction between the bait and prey fusions.

In another aspect, the invention provides a kit for detecting protein interactions, comprising:
(i) a first expression construct including a coding sequence for a targeting domain and a ligation site flanking an end of the targeting domain coding sequence for ligating a coding sequence of a bait polypeptide sequence in frame with said targeting domain coding sequence to produce a bait fusion protein, said first expression construct operably linked to a transcriptional regulatory element; and
(ii) a second expression construct including a coding sequence for a detection domain and a ligation site flanking an end of the detection domain coding sequence for ligating a coding sequence of a prey polypeptide sequence in frame with said detection domain coding sequence to produce a prey fusion protein, said second expression construct operably linked to a transcriptional regulatory element,

wherein, the targeting domain localizes the bait fusion protein to a subcellular location within a host cell, and the detection domain can be detected in a transcriptionally-independent manner in the host cell to determine if the prey fusion protein is associated in a complex with the bait fusion protein or not.

In yet another aspect, the invention provides a method for detecting protein interactions in a host cell, comprising:
(i) providing a host cell culture, the cells of which include:
   (a) a first nucleic acid coding sequence encoding a bait fusion protein comprising a bait polypeptide sequence fused to a targeting domain that targets the bait fusion protein to a subcellular location within the host cell, and
   (b) a second nucleic acid coding sequence encoding a prey fusion protein comprising a prey polypeptide sequence fused to one or more detection domains that can be detected in a transcriptionally-independent manner in the host cell to determine if the prey fusion protein is associated in a complex with the bait fusion protein or not,
   wherein the culture is a variegated mixture of cells containing different prey polypeptide sequences and/or different bait polypeptide sequences;
(ii) selecting cells from the culture in which the prey fusion protein is localized in the cell in the same subcellular pattern as the bait fusion protein;
(iii) identifying the sequence of the bait and prey fusion proteins from the selected cells

In another aspect, the invention provides a method for conducting a drug discovery business, comprising:
(i) using the methods of the invention to identify a protein complex for which an agent that inhibits or potentiates the formation or activity of the complex is desired;
(ii) generating a drug screening assay for identifying agents that inhibit or potentiate the formation or activity of the complex;
(iii) conducting animal toxicity profiles on a agent identified in step (ii), or an analog thereto;
(iv) manufacturing a pharmaceutical preparation of an agent having a suitable animal toxicity profile; and
(v) marketing the pharmaceutical preparation to healthcare providers.

In another aspect, the invention provides a method for conducting a drug discovery business, comprising:
(i) using the methods of the invention to identify a protein complex which is mediated by post-translational modification and for which an agent that inhibits or potentiates the post-translational modification is desired;
(ii) generating a drug screening assay for identifying agents that inhibit or potentiate the post-translational modification and effect the formation of the protein complex;
(iii) conducting animal toxicity profiles on a agent identified in step (ii), or an analog thereto;
(iv) manufacturing a pharmaceutical preparation of an agent having a suitable animal toxicity profile; and
(v) marketing the pharmaceutical preparation to healthcare providers.

In aother aspect, the invention provides a method for conducting a bioinformatics business, comprising:
(i) using the methods of the invention to identify networks of protein complexes;
(ii) generating a database including information identifying interactions of different proteins in a signal pathway and information identifying the proteins.

In aother aspect, the invention provides a system for analyzing protein complexes in cells, comprising a flow cytometer for analyzing cells and determining if a fluorescent signal is dispersed in a cell or localized to kinetochore structures. In certain embodiments, the invention may further comprise a microprocessor for comparing the flow spectra of cells and distinguishing between a diffuse pattern of fluorescence in the cells and a kinetochore-localized pattern.

In aother aspect, the invention provides a system for analyzing protein complexes in cells, comprising a microscope having a camera mounted therein for analyzing cells in a field of vision of the microscope, and a microprocessor for processing images obtained from said camera and determining if a fluorescent signal is dispersed in a cell or localized to kinetochore structures. In certain embodiments, the invention may further comprise a cell picking robot which is controlled by said microprocessor and isolates cells which the microprocessor has determined have a fluorescent signal localized to kinetochore structures.

### BRIEF DESCRIPTION OF THE FIGURES

FIG. 1 shows the subcellular localization of kinetochore proteins visualized using the CREST anti-kinetochore antibody.

FIG. 2 shows the targeting of myc-tagged human CENP-C to kinetchores in COS cells and Xenopus A6 cells transfected with the indicated CENP-C construct.

FIG. 3 shows the targeting of a CENP-C-beta-galactosidase fusion to the kinetochore.

FIG. 4 shows the targeting-dependent stability of CENP-C.

FIG. 5 shows the destabilization of beta-galactosidase by the CENP-C destruction box.

FIG. 6 is a schematic of the function of the CENP-C instability domain.

FIG. 7 shows the results of CENP-C expression plasmid dilution experiment indicating that a single vector can saturate the kinetochore.

FIG. 8 shows an exemplary design for a “bait” recombinant retrovirus construct.

FIG. 9 shows a schematic of the prey library construction and of the bait-prey interaction.

FIG. 10 shows a first exemplary method for FACS-aided screening for positive interactors.

FIG. 11 shows a second exemplary method for FACS-aided screening for positive interactors.

FIG. 12 is a schematic for a protein-protein interaction screen using optical approaches.

FIG. 13 is a schematic of the mammalian “three-hybrid system” to identify drug targets.

FIG. 14 is a schematic of the components for a mammalian three-hybrid system for drug screening.

FIG. 15 shows a schematic for use of a dimerization domain to enhance detection of bait/prey interactions.

FIG. 16 is a schematic of the incorporation of selective genetic markers into the prey construct to aid in the detection of protein-protein interactions.

FIG. 17 is the amino acid sequence for human CENP-C (GenBank Accession No. A46281).

FIG. 18 is the nucleotide sequence for human CENP-C (GenBank Accession No. NM001812.

### DETAILED DESCRIPTION OF THE INVENTION

1. General

This invention relates to in vivo methods for discovery and characterization of protein-protein and protein-ligand interactions in mammalian cells.

Small molecule-protein and protein-protein interactions have been shown to underlie the majority of signaling events in the cell. One prime example comes from one the earliest signaling pathways described at the molecular level, beta-adrenergic stimulation of glycogenolysis in hepatocytes (Sutherland E W, Science, 177: 401-408 (1972); Gilman A G, Harvey Lect, 85: 153-172 (1989)). Epinephrine secreted into the blood stream by the adrenals binds to high affinity beta-adrenergic receptors on the plasma membrane of hepatocytes. This interaction induces a conformational switch in the beta-adrenergic receptor such that this seven-transmembrane protein now acts as a nucleotide exchange factor for the guanine nucleotide binding protein, G-alpha-S. The nucleotide exchange function of the activated beta-adrenergic receptor causes G-alpha-S to release GDP and bind the more abundant GTP. G-alpha-S-GTP then activates adenylate cyclase resulting in the elevated production of cyclic AMP in the cell. Cyclic AMP then binds to and dissociates an inhibitory subunit of cyclic AMP-dependent protein kinase A from the catalytic subunit. Activated protein kinase A then phosphorylates downstream targets involved in glucose mobilization. These studies demonstrate that a single signaling pathway triggered by a small molecule ligand can involve multiple protein-protein and protein-ligand interactions that yield precise physiological responses in the cell. The significance of these kinds of interactions has been conclusively demonstrated in many other signaling pathways, a concept that forms a basis of our understanding of modern biology and medicine.

Given the fundamental importance of protein-protein and protein-ligand interactions in physiology, convenient methods for identifying and characterizing these events have been major goals of biological and pharmaceutical research. One such method, commonly known as “co-immunoprecipitation”, uses an antibody against a tester protein to isolate this protein from the complex mixture of proteins from cellular lysates, and then examines co-isolated proteins as candidate interacting species (Ewald S J and Refling P H, J Immunol, 134: 2513-19 (1985)). The obvious prerequisite for monospecific antibodies recognizing the tester protein precludes analysis of large numbers of tester proteins for their putative partners in the cell. Further, the identification of co-precipitating partner proteins requires direct protein sequencing techniques which, until the advent of mass spectroscopic techniques, proved insensitive and laborious.

Many of the problems faced by co-precipitation techniques in identifying protein-protein interactions were solved by the so-called “two-hybrid” system in yeast (Fields S and Song O, Nature, 340: 245-246 (1989); Mendelsohn A R and Brent R, Curr Opin Biotechnol, 5: 482-486 (1994); Vidal M, et al., Proc Natl Acad Sci USA, 93: 10315-10320 (1996)). In the two-hybrid system, interactions between a tester or “bait” protein and library of “prey” proteins are determined using genetic approaches yielding selectable changes in gene expression. Rather than requiring an antibody to a given tester protein, the cDNA encoding said protein is sufficient to establish the screen, and interacting proteins are also identified directly as cDNA sequences permitting analysis by direct DNA sequencing. Thus the two hybrid system, and permutations of the original design, has offered revolutionary advantages to identifying static, high affinity interactions.

While extremely powerful, the yeast two-hybrid system has two technological disadvantages. The first is that it is difficult to recapitulate physiological pathways relevant to mammalian signal transduction pathways. For instance, yeast lack beta-adrenergic receptors and therefore it is difficult to examine protein-protein interactions that only occur during such signaling. The second is that yeast is generally impermeable to small molecules such as cAMP rendering the use of such key compounds in signaling studies, as well as the performance of drug screens in general, difficult. Thus the otherwise efficient yeast two-hybrid system may be less optimal for identifying protein-protein interactions that occur as a consequence of particular physiological events in the cell. As these more transient interactions arguably comprise many of the regulated or conditional processes underlying physiological switches in the cell such interactions may be better addressed in a mammalian cell context where such pathways can be easily triggered. Several technologies have been developed to identify protein-protein interactions in the mammalian cell, including those modeled on the yeast two-hybrid detection system (Fotin-Mleczek M, et al., Biotechniques, 29: 22-26 (2000)), the beta-galactosidase alpha complementation system (Rossi F, et al., Proc Natl Acad Sci USA, 94: 8405-8410 (1997)), and fluorescence energy resonance transfer (FRET; Miyawaki A, et al., Nature, 388: 882-887 (1997)).

We have now discovered an animal cell screening system in which tester proteins have been targeted to specific subcellular sites. The system provides a convenient method for detecting protein-protein and protein-ligand interactions, including those which are of a transient nature dependent on activated signaling pathways. In addition, this system can be used in direct screens for protein-ligand interactions and in a reverse manner for discovering small molecules that disrupt protein-protein interactions. The invention provides a means for (1) targeting “bait” proteins or ligands to discrete and saturable sites within the cell, as well as technologies for limiting mistargeting of such bait molecules, (2) generating libraries of prey molecules with features that enhance their detection in the mammalian cell interaction system, (3) high-throughput screening of said libraries of prey molecules for interaction with a given bait, (4) screening of established interactions for small molecules or proteins which interfere with such binding, and (5) screening for proteins which interact with a known or orphaned ligand. The invention also features cell strains, genetic constructs, and detection approaches that facilitate the use of such a mammalian system for detecting interactions in a cellular context.

An essential element of this two-hybrid screen in animal cells is the patterning of the bait to a set of discrete foci whose pattern is unique and therefore easily recognizable. These foci should occupy a very small surface area as to be saturable with low levels of expressed prey protein. One example of a discrete set of subcellular sites is the kinetochore. The kinetochore is a multiprotein complex that assembles at the centromere of interphase and mitotic chromosomes. Given that each chromosome has one centromere, exactly 46 kinetochores exist in the nucleus of human cells. Investigations of chromosome segregation have revealed (Lanini L and McKeon F, Mol Biol Cell, 6: 1049-1059 (1995)) that CENP-C, a conserved kinetochore protein, faithfully targets to the kinetochores upon transfection of mammalian cells. Additionally, proteins fused to CENP-C, such as beta galactosidase, will also target to the kinetochore along with CENP-C. Therefore any protein can be ectopically positioned to the kinetochore as a fusion with CENP-C. As important, it appears that efficient labeling of the kinetochore with the bait-CENP-C fusion protein requires only a single expression plasmid, a situation not altered by introducing as many as 30 additional copies. This observation may be germane to one of the some of the most vexing problems in establishing a single cell screen, including sensitivity and signal-to-noise. CENP-C is therefore an exemplary candidate for this platform for two reasons. First, its focal but limited binding sites at the kinetochore permits detection and saturation of the signal at low expression levels. Second, CENP-C that fails to target to the kinetochore is rapidly degraded in the cell. Therefore the precise labeling of the kinetochore is upheld at all CENP-C levels, resulting in maintenance of uniform bait localization.

Requirements for the prey library include rapid deconvolution of positives from single cells or clones derived from single cells. In an exemplary embodiment, a CENP-C fusion with the desired bait protein is constructed in a retroviral vector and a cell line established by transduction and selection for a resistance marker. The targeting of the CENP-C-fusion protein to the kinetochore of this host cell line will be determined by standard immunofluorescense. The prey library, also in a retroviral vector, is introduced by superinfection. The prey library construction takes into account a means of rapidly determining sequence identity of the encoded cDNA from a single cell or a clone derived therefrom, a process which depends on RT-PCR from vector-specific sequences.

The ability to detect interactions using a single plasmid permits the use of standard retroviral vectors for expressing prey constructs. Retroviruses efficiently transduce most cell lines but in general only one virus is incorporated into a given cell before antiviral mechanisms triggered in the cell prevent superinfection. While a single vector is not useful for many cell-based assays, in the CENP-C system the bait protein is concentrated at a particular subcellular location (e.g., a CENP-C-bait fusion at the centromere) permits the detection of the expression product of the single incorporated virus. Therefore the traditional weakness of the retroviral system is converted, by the present system, into a strength in that each cell represents a single assay of interactions between the bait and a given prey protein. Given the high transduction efficiency of the retroviruses and the fact that a typical assay on 22 mm coverslips employs approximately 250,000 cells, an entire library of one million cDNA clones may be assayed on a surface the size of a typical microscope slide. Additionally, high-throughput optical scanning of cells may be used in conjunction with FACS analysis to sort cells to 96 well culture tubes for development of cell clones for prey analysis and follow-up studies of drug discovery.

2. Definitions

For convenience, certain terms employed in the specification, examples, and appended claims are collected here.

The term “agonist” as used herein, refers to a molecule which augments formation of a protein complex or which increases the amount of, or prolongs the duration of, the activity of a protein. Agonists may include proteins, nucleic acids, carbohydrates, or any other molecules that bind to a protein complex or a molecule of the complex. Peptide mimetics, synthetic molecules with physical structures designed to mimic structural features of particular peptides, may serve as agonists. The stimulation may be direct, or indirect, or by a competitive or non-competitive mechanism.

As used herein the term “animal” refers to mammals, preferably mammals such as humans.

The term “antagonist”, as used herein, refers to a molecule which, when bound to a protein or protein complex decreases the amount of or duration of the activity of the protein complex, or a protein member thereof, or decreases complex formation. Antagonists include compounds that directly inhibit the activity of a protein in the complex. Antagonists may include proteins such as antibodies that compete for binding at a binding region of a complex member, nucleic acids, including anti-sense molecules that arrest expression of a complex member at the genetic level, carbohydrates, or any other molecules that bind to a protein of interest to an extent efficient for preventing complex formation or activity. Antagonists also include dominant negative mutatnts, e.g. a member of a complex that contains a mutated active site. Antagonists further include a peptide or peptide fragment derived from a protein complex member, but will not include the full-length sequence of the wild-type molecule. Peptide mimetics, synthetic molecules with physical structures designed to mimic structural features of particular peptides, may serve as antagonists. The inhibition may be direct, or indirect, or by a competitive or non-competitive mechanism.

The terms “bait” or “bait protein” refer to a polypeptide that is used as a target to find other proteins which may associate with it. Typically, a bait protein is tagged or immobilized so as to allow easy isolation of complexes involving the bait protein.

The term “binding” refers to a stable association between two molecules, in the present case between a bait protein and a binding partner, such as another polypeptide or a protein substrate, due to, for example, electrostatic, hydrophobic, ionic and/or hydrogen-bond interactions under physiological conditions.

“Cells,” “host cells”or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding a polypeptide with a second amino acid sequence defining a domain foreign to and not substantially homologous with any domain of the protein. A chimeric protein may present a foreign domain that is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, and natural product extract libraries.

In certain instances, the terms “homologous” or “homolog thereof” are used. Many of the components of the bait and prey fusion proteins, such as the targeting domain, the detection domain, instability domains, rescue sequences and oligomerization domains are preferably derived from wild-type proteins. However, as will be apparent to the skilled artisan, the subject assay can be carried using bait or prey fusion proteins in which these domains having amino acid sequences which are homologous, but not identical, to the wild-type sequence yet retain the desired activity. In preferred embodiments, a “homologous” sequence is one which can be encoded by a nucleic acid which hybridizes to a wild-type coding sequence under wash conditions of 1.0×SSC at 65° C., and even more preferably under wash conditions of 0.2×SSC at 65° C. A “homologous” sequence can also be one which has an amino acid sequence at least 80 percent identical to a wild-type sequence, and even more preferably at least 90 or 95 percent identical.

The phrases “conserved residue” “or conservative amino acid substitution” refer to groupings of amino acids on the basis of certain common properties. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.
   In addition to the groups presented above, each amino acid residue may form its own group, and the group formed by an individual amino acid may be referred to simply by the one and/or three letter abbreviation for that amino acid commonly used in the art.

The term “construct” refers to a nucleotide or amino acid sequence which contains at least two component parts fused together in tandem.

The terms “destruction sequence”, “destruction domain”, “instability sequence” and “instability domain” are used herein interchangeably and are meant to refer to an sequence of amino acids capable of conferring the degredation of the polypeptide comprising the sequence. The destruction or instability sequence may be a protein, protein fragment or peptide derived from a naturally occuring polypeptide or may be an artificial sequence.

The term “detection sequence” as used herein refers to a protein, protein fragment or peptide which permits detection of the polypeptide comprising the sequence. The detection sequence may allow direct detection of the polypeptide without the need for additional components (e.g., without needing to add additional reagents such as substrates, antibodies, etc.), for example a fluorescent or luminescent polypeptide. Alternatively, the detection sequence may provide a means for indirect detection requiring a substrate, such as beta-lactamase, or an antibody, such as for recognition of an epitope.

The term “DNA sequence encoding a polypeptide” may refer to one or more genes within a particular individual. As is well known in the art, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. Moreover, certain differences in nucleotide sequences may exist between individual organisms, which are called alleles. Such allelic differences may or may not result in differences in amino acid sequence of the encoded polypeptide yet still encode a protein with the same biological activity.

The term “domain” as used herein refers to a region within a protein that comprises a particular structure or function different from that of other sections of the molecule.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding a polypeptide and comprising exon coding sequences, though it may optionally include intron sequences derived from a chromosomal gene. The term “intron” refers to a DNA sequence present in a given gene which is not translated into protein and is generally found between exons.

The term “isolated”, as used herein with reference to a protein or protein complex, refers to a protein or protein complex that is essentially free from contaminating proteins that normally would be present in cellular milieu in which the protein occurs or the complex forms endogenously. Thus, an isolated an protein or protein complex is isolated from cellular components that normally would “contaminate” or interfere with the study of the protein or protein complex in isolation, for instance while screening for modulators thereof. It is to be understood, however, that such an “isolated” protein or protein complex may incorporate other proteins the modulation of which is being investigated.

The term “isolated” as also used herein with respect to nucleic acids, such as DNA or RNA, refers to molecules separated from other DNAs, or RNAs, respectively, that are present in the natural source of the macromolecule. For example, isolated nucleic acids encoding a polypeptide preferably include no more than 10 kilobases (kb) of nucleic acid sequence which naturally immediately flanks a particular gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

The term “motif” as used herein refers to an amino acid sequence that is commonly found in a protein of a particular structure or function. Typically a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases such as GenBank or SwissProt can be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The terms peptides, proteins and polypeptides are used interchangeably herein.

The term “prey” refers to a test molecule which is being assayed for its ability to interact with a bait molecule.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

The term “rescue sequence” as used herein refers to a protein, protein fragment or peptide which permits identification, purification or isolation of the polypeptide comprising the sequence. Exemplary rescue sequences included epitotpe tags such as the His-tag or myc-tag.

The term “selection sequence” as used herein refers to a protein, protein fragment or peptide which confers a growth advantage to a cell expressing the sequence. Exemplary selection sequences include antibiotic resistance proteins or proteins required to complement an auxotrophic phenotype. Preferably, the selection sequence permits isolation of a cell, or population of cells, expressing the selection sequence based on an increased ability to grow under selective conditions as compared to cells not expressing the selection sequence.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fingal, bacterial, or algal extracts, which can be screened with any of the assays of the invention.

The term “targeting sequence” and “subcellular localization domain” are used herein interchangeably and refer to a protein, protein fragment or peptide capable of targeting the polypeptide comprising the target sequence to a particular location within the cell.

As used herein, the term “transfection” means the introduction of a nucleic acid, e.g., an expression vector, into a recipient cell by nucleic acid-mediated gene transfer.

As used herein, the term “vector” refers to a nucleic acid molecule capable of transporting another nucleic acid to which it has been linked. Vectors capable of directing the expression of genes to which they are operatively linked are referred to herein as “expression vectors”.

3. Bait Protein Constructs

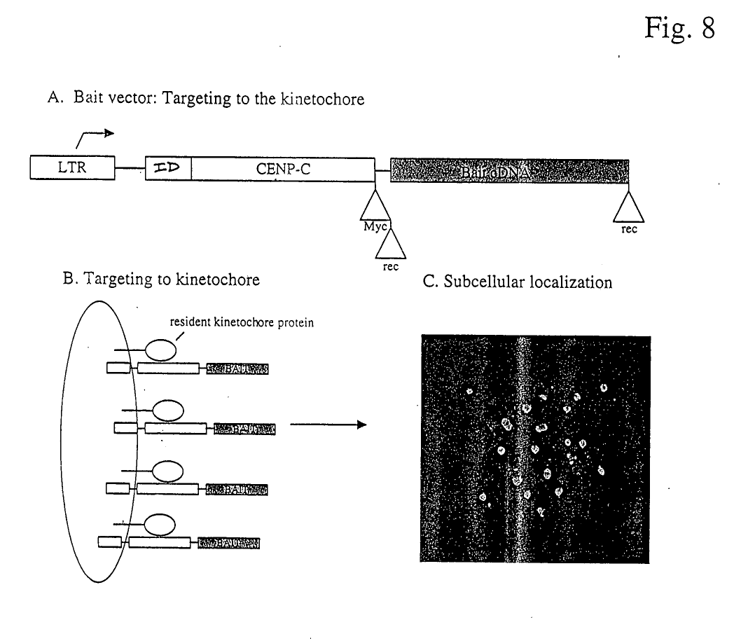

One of the first steps of the two-hybrid system of the present invention is construction of the bait fusion protein. To do this, sequences encoding a protein of interest or a polypeptide library are cloned in-frame to a targeting sequence that encodes for a domain or motif capable of targeting the protein construct to a particular location within the cell (FIG. 8). The localization of proteins within a cell is a simple method for increasing the effective concentration of the protein and thereby increasing the sensitivity of the two hybrid methods of the invention. Additionally, localization to (a) discreet subcellular site(s) serves as a means to assay for specifically interacting “prey” proteins. In preferred embodiments the bait protein construct may also contain at least one of an instability (or degradation) domain, a rescue tag and/or a selection tag.

The bait portion of the bait fusion protein may be chosen from any protein of interest and includes proteins of unknown, known, or suspected diagnostic, therapeutic, or pharmacological importance. The bait portion may also be chosen from protein fragments, peptides, modified or mutant proteins, etc. Exemplary bait proteins include, but are not limited to, oncoproteins (such as myc, particularly the C-terminus of myc, ras, src, fos, and particularly the oligomeric interaction domains of fos), tumor-suppressor proteins (such as p53, Rb, INK4 proteins [p16INK4a, p15INK4b], CIP/KIP proteins [p21CIP1, p27KIP1]) or any other proteins involved in cell-cycle regulation (such as kinases and phosphatases). In other embodiments, the bait polypeptide can be generated using all or a portion of a protein involved in signal transduction, including such motifs as SH2 and SH3 domains, ITAMs, ITIMs, kinase, phospholipase, or phosphatase domains, cytoplasmic tails of receptors and the like. Yet other preferred bait fusion proteins are generated with cytoskeletal proteins or factors involved in transcription or translation, or portions thereof. Still other bait fusion proteins can be generated with viral proteins.

In preferred embodiments, where the bait protein includes a catalytic domain of an enzyme, the fusion protein is derived with a catalytically inactive mutant, most preferably a mutant that binds substrate with about the Kₘ of the wild-type enzyme but with a greatly diminished K_{cat} for the catalyzed reaction with the substrate. For example, mutation of a residue in the catalytic site of the enzyme can give rise to such catalytically inactive mutants. Particular examples include point mutation of the active site lysine of a kinase, the active site serine of a serine protease or the active site cysteine of a phosphatase. Thus, the binding of the bait polypeptide portion of the fusion protein to a polypeptide substrate presented by a prey fusion protein can be enhanced. In each case, the protein of interest is fused to a targeting domain.

The targeting domain is a protein, protein fragment or peptide which confers on the bait construct the ability to localize to a specific, predetermined subcellular site within the cell. Preferably, the targeting domain is capable of docking the bait to a discreet subcellular location(s) having a distinctive pattern of localization. For example, mammalian cells contain exactly 46 kinetochores so that proteins capable of localizing to the kinetochores may be characterized by the formation of 46 discrete areas of localization within the cell. Preferred subcellular locations, and proteins capable of localizing to these discreet sites include, but are not limited to:

Nuclear sites, such as nucleoli (using nucleolar-localization signals); telomeres; kinetochores (using a variety of proteins, including CENP-A (GenBank Accession No. NM_{—}001800), CENP-B (GenBank Accession No. CAA38879), CENP-C (GenBank Accession No. A46281), CENP-E (GenBank Accession No. NM 001804), CENP-F (GenBank Accession No. NP_{—}057427), Bub1 (GenBank Accession No. AAD43675), Bub3 (GenBank Accession No. AAC06258), MAD3L (GenBank Accession No. AAC06260), and MAD2 (GenBank Accession No. AAC50781), etc.); nuclear envelope (using proteins including lamin A, lamin B, lamin C, emerins, porins, etc.); and other nuclear proteins or localization domains capable of targeting the bait to discrete subcellular locations on chromosomes or chromatin;

Cytoplasmic sites and organelles including the endoplasmic reticulum, Golgi, centrosome, tansgolgi network, cytoplasmic vesicles, mitochondria, secretory vesicles, lysosome, etc.; and

Membrane bound structures including plasma membranes, intracellular membrane vesicles, nuclear membranes, synapses, basolatemal membranes, etc.

Specific examples of targeting sequences which may be used in accord with the invention are given in Tables 1-6 and are further described below.

In one embodiment, the targeting sequence is a nuclear localization signal (NLS). NLSs are generally short, positively charged (basic) domains that serve to direct the entire protein in which they occur to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-beta nuclear localization signal (ARRRRP); NF-kappa-B p50 (EEVQRKRQKL); Ghosh et al., Cell 62:1019 (1990); NF-kappa-B p65 (EEKRKRTYE); Nolan et al., Cell 64:961 (1991); and others (see for example Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458,1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. U.S.A.,84:6795-6799, 1987; Galileo, et al., Proc. Natl. Acad. Sci. U.S.A., 87:458-462, 1990.

TABLE 1

Exemplary proteins containing targeting sequences capable of conferring nuclear
localization.

| | | | |
|---|---|---|---|
| Protein | Targeting Sequence | Reference | SEQ ID NO |

| | | |
|---|---|---|
| SV40 (monkey | PKKKRKV | Kalderon (1984), et al., |
| virus) large T | | Cell, 39:499-509 |
| Antigen | | |
| human retmoic acid | ARRRRP | |
| receptor-beta | | |
| NE-kappa-B p50 | EEVQRKRQKL | Ghosh et al., Cell |
| | | 62:1019 (1990) |
| NE-kappa-B p65 | EEKRKRTYE | Nolan et al., Cell 64:961 |
| | | (1991) and Boulikas, J. |
| | | Cell. Biochem. 55(1):32- |
| | | 58 (1994) |
| Xenopus | AVKRPAATKKAGQAKKKKLD | Dingwall, et al., Cell, |
| nucleoplasmin | | 30:449-458, 1982 and |
| protein | | Dingwall, et al., J. Cell |
| | | Biol., 107:641-849; 1988 |
| CENP-C | amino acids 373-943 of GenBank | |
| | Accession No. A46281 | |

In another embodiment, the targeting sequence is a membrane anchoring signal sequence. Membrane-anchoring sequences are well known in the art and are based on the genetic geometry of mammalian transmembrane molecules. Peptides are inserted into the membrane based on a signal sequence (designated herein as ssTM) and require a hydrophobic transmembrane domain (herein TM). The transmembrane proteins are inserted into the membrane such that the regions encoded 5′ of the transmembrane domain are extracellular and the sequences 3′ become intracellular. Of course, if these transmembrane domains are placed 5′ of the variable region, they will serve to anchor it as an intracellular domain, which may be desirable in some embodiments. ssTMs and TMs are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

As will be appreciated by those in the art, membrane-anchoring sequences, including both ssTM and TM, are known for a wide variety of proteins and any of these may be used. Particularly preferred membrane-anchoring sequences include, but are not limited to, those derived from CD8, ICAM-2, IL-8R, CD4 and LFA-1.

Useful sequences include sequences from: 1) class I integral membrane proteins such as IL-2 receptor beta-chain (residues 1-26 are the signal sequence, 241-265 are the transmembrane residues; see Hatakeyama et al., Science 244:551 (1989) and von Heijne et al, Eur. J. Biochem. 174:671 (1988)) and insulin receptor beta chain (residues 1-27 are the signal, 957-959 are the transmembrane domain and 960-1382 are the cytoplasmic domain; see Hatakeyama, supra, and Ebina et al., Cell 40:747 (1985)); 2) class II integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (MASPLTRFLSLNLLLLGESILGSGEAKPQAP; Nakauchi et al., PNAS U.S.A. 82:5126 (1985) and 1-21 in the case of ICAM-2 (MSSFGYRTLTVALFTLICCPG; Staunton et al., Nature (London) 339:61 (1989)). These leader sequences deliver the construct to the membrane while the hydrophobic transmembrane domains, placed 3′ of the random peptide region, serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR; Nakauchi et al., PNAS U.S.A. 82:5126 (1985)) and 224-256 from ICAM-2 (MVIIVTVVSVLLSLFVTSVLLCFIFGQHLRQQR; Staunton et al., Nature (London) 339:61 (1989)).

Alternatively, membrane anchoring sequences include the GPI anchor, which results in a covalent bond between the molecule and the lipid bilayer via a glycosyl-phosphatidylinositol bond for example in DAF (PNKGSGTTSGTTRLLSGHTCFTLTGLLGTLVTMGLLT; see Homans et al., Nature 333(6170):269-72 (1988), and Moran et al., J. Biol. Chem. 266:1250 (1991)). In order to do this, the GPI sequence from Thy-1 can be inserted 3′ of the variable region in place of a transmembrane sequence.

Similarly, myristylation sequences can serve as membrane anchoring sequences. It is known that the myristylation of c-src recruits it to the plasma membrane. This is a simple and effective method of membrane localization, given that the first 14 amino acids of the protein are solely responsible for this function: MGSSKSKPKDPSQR (see Cross et al., Mol. Cell. Biol. 4(9):1834 (1984); Spencer et al., Science 262:1019-1024 (1993), both of which are hereby incorporated by reference). Other modifications such as palmitoylation can be used to anchor constructs in the plasma membrane; for example, palmitoylation sequences from the G protein-coupled receptor kinase GRK6 sequence (LLQRLFSRQDCCGNCSDSEEELPTRL, Stoffel et al., J. Biol. Chem 269:27791 (1994)); from rhodopsin (KQFRNCMLTSLCCGKNPLGD; Barnstable et al., J. Mol. Neurosci. 5(3):207 (1994)); and the p21 H-ras 1 protein (NPPDESGPGCMSCKCVLS; Capon et al., Nature 302:33 (1983)).

Furthermore, farnesylation sequences (for example, P21 H-ras 1; LNPPDESGPGCMSCKCVLS; Capon, supra); and geranylgeranylation sequences (for example, protein rab-5A; LTEPTQPTRNQCCSN, geranylgeranylated; Farnsworth, PNAS U.S.A. 91:11963 (1994)) can serve as membrane anchoring sequences.

TABLE 2

Exemplary proteins containing a targeting sequence capable of
conferring membrane localizing.

| | | | |
|---|---|---|---|
| Protein | Targeting Sequence | Reference | SEQ ID NO |

| | | |
|---|---|---|
| CD8 | MASPLTRFLSLNLLLLGESILGSGE | Nakauchi et al., PNAS |
| | AKPQAP | U.S.A. 82:5126 (1985) |
| ICAM-2 | MSSFGYRTLTVALFTLICCPG | Staunton et al., Nature |
| | | (London) 339:61 (1989) |
| CD8 | PQRPEDCRPRGSVKGTGLDFACD | Nakauchi et al., PNAS |
| | IYLWAPLAGICVALLLSLIITLICYHSR | U.S.A. 82:5126 (1985) |
| ICAM-2 | MVIIVTVVSVLLSLFVTSVLLCFIF | Staunton et al., Nature |
| | GQHLRQQR | (London) 339:61 (1989) |
| DAF | PNKGSGTTSGTTRLLSGHTCFTLT | Homans et al., Nature |
| | GLLGTLVTMGLLT | 333(61 70):269-72 |
| | | (1988), and Moran et al., |
| | | J. Biol. Chem. 266:1250 |
| | | (1991) |
| c-src | MGSSKSKPKDPSQR | Cross et al., Mol. Cell. |
| | | Biol. 4(9):1834 (1984); |
| | | Spencer et al., Science |
| | | 262:1019-1024 (1993) |
| GRK6 | LLQRLFSRQDCCGNCSDSEEELPTRL | Stoffel et al., J. Biol. |
| | | Chem 269:27791 (1994) |
| rhodopsin | KQFRNCMLTSLCCGKNPLGD | Barnstable et al., J. Mol. |
| | | Neurosci. 5(3):207 |
| | | (1994) |
| p21 H-ras 1 | LNPPDESGPGCMSCKCVLS | Capon et al., Nature |
| | | 302:33 (1983) |
| rab-5A | LTEPTQPTRNQCCSN | Farnsworth, PNAS |
| | | U.S.A. 91:11963 (1994) |

In yet another embodiment, the targeting sequence is a lysozomal targeting sequence, including, for example, a lysosomal degradation sequence such as Lamp-2 (KFERQ; Dice, Ann. N.Y. Acad. Sci. 674:58 (1992); or lysosomal membrane sequences from Lamp-1 (MLIPIAGFFALAGLVLIVLIAYLIGRKRSHAGYOTI), Uthayakumar et al., Cell. Mol. Biol. Res. 41:405 (1995)) or Lamp-2 (LVPIAVGAALAGVLILVLLAYFIGLKHHHAGYEQF, Konecki et la., Biochem. Biophys. Res. Comm. 205:1-5 (1994)).

TABLE 3

Exemplary proteins containing a targeting sequence capable
of conferring lysozomal localization.

| | | | |
|---|---|---|---|
| Protein | Targeting Sequence | Reference | SEQ ID NO |

| | | |
|---|---|---|
| Lamp-2 | KFERQ | Dice, Ann. N.Y. Acad. |
| | | Sci. 674:58 (1992) |
| Lamp-1 | MLIPIAGFFALAGLVLIVLIAYL | Uthayakumar et al., Cell. |
| | IGRKRSHAGYOTI | Mol. Biol. Res. 41:405 |
| | | (1995) |
| Lamp-2 | LVPIAVGAALAGVLIVLLAYFIG | Konecki et al., Biochem. |
| | LKHHHAGYEQF | Biophys. Res. Comm. |
| | | 205:1-5 (1994) |

Alternatively, the targeting sequence may be a mitrochondrial localization sequence, including mitochondrial matrix sequences (e.g., yeast alcohol dehydrogenase III; MLRTSSLFTRRVQPSLFSRNILRLQST; Schatz, Eur. J. Biochem. 165:1-6 (1987)); mitochondrial inner membrane sequences (yeast cytochrome c oxidase subunit IV; MLSLRQSIRFFKPATRTLCSSRYLL; Schatz, Eur. J. Biochem. 165:1-6 (1987)); mitochondrial intermembrane space sequences (yeast cytochrome c1; MFSMLSKRWAQRTLSKSFYSTATGAASKSGKLTQKLVTAGVAAAGITASTLLYAD SLTAEAMTA; Schatz, Eur. J. Biochem. 165:1-6 (1987)) or mitochondrial outer membrane sequences (yeast 70 kD outer membrane protein; MKSFITRNKTAILATVAATGTAIGAYYYYNQLQQQQQRGKK; Schatz, Eur. J. Biochem. 165:1-6 (1987)).

TABLE 4

Exemplary proteins containing a targeting sequence
capable of conferring mitochondrial localization.

| | | | |
|---|---|---|---|
| Protein | Targeting Sequence | Reference | SEQ ID NO |

| | | |
|---|---|---|
| yeast alcohol | MLRTSSLFTRRVQPSLFSRNILR | Schatz, Eur. J. |
| dehydrogenase | LQST | Biochem. 165:1-6 |
| III | | (1987) |
| yeast | MLSLRQSIRFFKPATRTLCSSRYLL | Schatz, Eur. J. |
| cytochrome c | | Biochem. 165:1-6 |
| oxidase | | (1987) |
| subunit IV | | |
| yeast | MFSMLSKRWAQRTLSKSFYSTATG | Schatz, Eur. J. |
| cytochrome c1 | AASKSGKLTQKLVTAGVAAAGITA | Biochem. 165:1-6 |
| | STLLYADSLTAEAMTA | (1987) |
| yeast 70 kD | MKSFITRNKTAILATVAATGTAIGA | Schatz, Eur. J. |
| outer | YYYYNQLQQQQQRGKK | Biochem. 165:1-6 |
| membrane | | (1987) |
| protein | | |

The target sequences may also be capable of localizing the bait construct to the endoplasmic reticulum. Proteins capable of inducing localization to the endoplasmic reticulum include, for example, the sequences from calreticulin (KDEL; Pelham, Royal Society London Transactions B; 1-10 (1992)) or adenovirus E3/19K protein (LYLSRRSFIDEKKMP; Jackson et al., EMBO J. 9:3153 (1990).

TABLE 5

Exemplary proteins containing a targeting sequence
capable of conferring endoplasmic reticulum
(ER) localization.

| | | | |
|---|---|---|---|
| Protein | Targeting Sequence | Reference | SEQ ID NO |

| | | |
|---|---|---|
| calreticulin | KDEL | Pelham, Royal Society |
| | | London Transactions B; |
| | | 1-10 (1992) |
| E3/19K | LYLSRRSFIDEKKMP | Jackson et al., EMBO J. |
| protein | 9:3 153 (1990) | |

Furthermore, targeting sequences also include peroxisome sequences (for example, the peroxisome matrix sequence from Luciferase; SKL; Keller et al., PNAS U.S.A. 4:3264 (1987)).

TABLE 6
Exemplary proteins containing a targeting sequence capable
of conferring peroxisome localization.

| | | | SEQ ID |
|---|---|---|---|
| Protein | Tageting Sequence | Reference | NO |
| peroxisome | SKL | Keller et al., PNAS | |
| matrix sequence | | U.S.A. 4: 3264 (1987) | |
| from Luciferase | | | |

In a particularly preferred embodiment, the target sequence is the CENP-C protein, or a portion thereof, which is capable of localizing to the kinetochores. Preferably, the target sequence comprises at least amino acids 373-943 of the CENP-C protein sequence (GenBank Accession Number A4628 1).

In addition to the targeting sequences described herein, determination of alternative targeting sequences would be easily obtainable by the skilled artisan without undue experimentation. For example, one or more potential targeting sequences could be attached to a detector sequence (e.g., GFP or the like) and introduced into a host cell. The ability of the targeting sequence to cause specific localization of GFP could then be assayed using optical techniques to determine the location of the GFP within the cell. A library of potential targeting sequences, either from naturally occurring sequences or engineered sequences, could rapidly be screened using such a method.

In a preferred embodiment, the bait construct additionally contains an instability or destruction sequence (see Table 7). The instability sequence may be a protein, protein fragment or peptide that is capable of causing degradation of the bait construct when not localized to the desired subcellular location. A variety of amino acid sequences capable of directing protein degradation are known to the skilled artisan, including, for example, a destruction sequence as found in cyclin B1, RTALGDIGN, Klotzbucher et al., EMBO J. 1:3053 (1996); a lysosomal degradation sequence such as found in Lamp-2, KFERQ, Dice, Ann. N.Y. Acad. Sci. 674:58 (1992); a destruction box sequence, RxxLxxxxN, Glotzer et al. (1991) Nature 349:132-138; a PEST sequence, which is a region of an amino acid sequence enriched for proline (P), glutamate (E), serine (S), and threonine (T) residues (PEST) in no particular order, Rechsteiner and Rogers, 1996, TIBS 21:267-271; and an F-box sequence, ZJXZPZUZZXXZZXXXXXXXZZXZXXVXBBZXXZZXXX-XZOXXZ, wherein Z is a nonpolar amino acid residue (ala, val, leu, iso, pro, phe, met, trp), X is any amino acid residue, B is a basic amino acid residue (lys, arg, his), U is an acidic amino acid residue (asp, glu), O is an aromatic amino acid residue (phe, tyr, trp), J is either serine or threonine (ser, thr), and P and V are the standard single letter representations for proline and valine, respectively, Craig and Tyers, Prog. Biophys. & Mol. Biol. 72: 299-328 (1999). Particularly preferred is the instability domain of CENP-C comprising at least amino acids 249-323 of the CENP-C protein sequence.

TABLE 8
Exemplary proteins containing a destruction sequence and consensus destruction
sequences capable of targeting a protein for degradation.

| Protein/consensus | Destruction Sequence | Reference | SEQ ID NO |
|---|---|---|---|
| cyclin B1 | RTALGDIGN | Klotzbucher et al., | |
| | | EMBO J. 1: 3053 (1996) | |
| Lamp-2 | KFERQ | Dice, Ann. N.Y. Acad. | |
| | | Sci. 674: 58 (1992) | |
| Destruction Box | RxxLxxxxN* | Glotzer et al. (1991) | |
| consensus | | Nature 349: 132-138 | |
| F-box consensus | ZJxZPZUZZxxZZxxxxxxxZZxZxx | Craig and Tyers, Prog. | |
| | VxBBZxxZZxxxxZOxxZ* | Biophys. & Mol. Biol. | |
| | | 72: 299-328 (1999) | |
| PEST consensus | Sequences enriched in proline (P), | Rogers, et al., (1986) | |
| sequence | glutamic acid (E), serine (S) and | Science 234, 364-368 | |
| | threonine (T) | and Rechsteiner and | |
| | | Rogers, 1996, TIBS | |
| | | 21: 267-271 | |
| CENP-C | amino acids 249-323 of GenBank | | |
| | Accession No. A46281 | | |

*wherein Z is a nonpolar amino acid residue (ala, val, leu, iso, pro, phe, met, trp), x is any amino acid residue, B is a basic amino acid residue (lys, arg, his), U is an acidic amino acid residue (asp, glu), O is an aromatic amino acid residue (phe, tyr, trp), J is either serine or threonine (ser, thr), and P and V are the standard single letter representations for proline and valine, respectively

As described above for the targeting sequence, determination of instability sequences useful in the methods of the invention would be easily obtainable by the skilled artisan without undue experimentation. For example, one or more potential instability sequences could be attached to a detector sequence (e.g., GFP or the like) and introduced into a host cell. The ability of the instability sequence to cause degradation of the GFP construct could be easily assayed simply by detecting the presence of GFP expression within the cell. A library of potential target sequences, either from naturally occurring sequences or non-naturally occurring sequences, could rapidly be screened using such a method. Additionally, a similar screen combining a known or potential targeting sequence and a known or potential instability sequence attached to GFP could be carried out to determine if the instability sequence can confer degradation of the fusion construct only when not correctly targeted to a specific subcellular localization.

In another preferred embodiment, the bait construct additionally contains a selection gene. Selection genes allow the selection of transformed host cells containing the bait construct, and particularly in the case of mammalian cells, ensures the stability of the vector, since cells which do not contain the vector will generally die. Selection genes are well known in the art and will vary with the host cell used. Suitable selection genes include, but are not limited to, neomycin, blastocidin, bleomycin, puromycin, hygromycin, and other drug resistance genes. In some cases, for example when using retroviral vectors, the requirement for selection genes is lessened due to the high transformation efficiencies that can be achieved. Accordingly, selection genes need not be used in retroviral constructs, although they can be. In addition, when retroviral vectors are used, the bait construct may also contain a detection sequence, as described below, rather than a selection sequence. It may be desirable to verify that the vector is present in the cell, but not require selective pressure for maintenance. Preferably, the selection sequence on the bait construct will be different from that on the prey construct, although in certain embodiments, the same selection sequence may be used on both constructs.

In yet another preferred embodiment, the bait construct additionally contains a rescue sequence. A rescue sequence is a sequence that may be used to identify, purify or isolate either the bait protein construct or the nucleic acid encoding it. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, GST, and Strep tag I and II. Alternatively, the rescue sequence may be a unique oligonucleotide sequence which serves as a probe target site to allow the quick and easy isolation of the bait construct, via PCR, related techniques, or hybridization.

The use of recombinant DNA techniques to create a fusion construct, with the translational product being the desired bait fusion protein, is well known in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, Eds. Ausubel et al. John Wiley & Sons: 1992).

The components of the bait construct may be arranged in any order which allow it to function as desired in the two hybrid methods of the invention. It may be necessary in some instances to introduce an unstructured polypeptide linker region between one or more of the components of the bait protein construct. The linker can facilitate enhanced flexibility of the fusion protein allowing the localization domain to freely make inter-protein contacts. The linker can also reduce steric hindrance between the components, and allow appropriate interaction of the bait polypeptide portion with a prey polypeptide component of the two-hybrid system. The linker can also facilitate the appropriate folding of each component to occur. The linker can be of natural origin, such as a sequence determined to exist in random coil between two domains of a protein. Alternatively, the linker can be of synthetic origin. For instance, the sequence (Gly₄Ser)₃ can be used as a synthetic unstructured linker. Linkers of this type are described in Huston et al. (1988) PNAS 85: 4879; and U.S. Pat. No. 5,091,513, both incorporated by reference herein. Another exemplary embodiment includes a poly alanine sequence, e.g., (Ala)₃.

4. Prey Protein Constructs

The present invention also provides prey vectors. Generally, the prey vector is a distinct vector from the bait vector, although as will be appreciated by those in the art, one or two independent vectors may be used. That is, the components of the bait and prey vectors could reside on a single vector or on two vectors. Generally, when the prey protein is a member of a library, as is outlined below, the prey vector will be separate from the bait vector.

The prey vector or construct contains a prey protein fused in-frame to a detector protein (e.g., GFP or the like). If a protein-protein interaction occurs between the bait and prey proteins, the prey protein will become localized to the specific subcellular site to which the bait was targeted. The prey protein may then be assayed using manual or automated optical detection methods to determine the pattern of localization of the prey protein. The prey construct may also optionally contain one or more of an instability sequence, a rescue sequence, a selection sequence, and/or a dimerization sequence.

“Prey protein,” as used herein, is meant to indicate a candidate protein that is to be tested for interaction with a bait protein. Protein in this context means proteins, oligopeptides, and peptides, i.e. a sequence of at least two amino acids. In a preferred embodiment, the prey protein sequence is one of a library of prey protein sequences; that is, a library of prey proteins is tested for binding to one or more bait proteins. The prey protein sequences can be derived from genomic DNA, cDNA or can be random sequences. Alternatively, specific classes of prey proteins may be tested. The library of prey proteins or sequences encoding prey proteins are incorporated into a library of expression vectors, each or most containing a different prey protein sequence.

In a preferred embodiment, the prey protein sequences are derived from genomic DNA sequences. Generally, as will be appreciated by those in the art, genomic digests are cloned into prey vectors. The genomic library may be a complete library, or it may be fractionated or enriched as will be appreciated by those in the art.

In a preferred embodiment, the prey protein sequences are derived from cDNA libraries. A cDNA library from any number of different cells may be used, and cloned into prey vectors. As above, the cDNA library may be a complete library, or it may be fractionated or enriched in a number of ways.

In a preferred embodiment, the prey protein sequences are random sequences. Generally, these will be generated from chemically synthesized oligonucleotides. Generally, random prey proteins range in size from about 2 amino acids to about 100 amino acids, with from about 10 to about 50 amino acids being preferred. As above, fully random or “biased” random proteins may be used.

It will be appreciated by those skilled in the art that many variations of the prey and bait fusion proteins can be constructed and should be considered within the scope of the present invention. For example, it will be understood that, for screening polypeptide libraries, the identity of the prey polypeptide can be fixed and the bait protein can be varied to generate the library. Indeed, in certain embodiments it will be desirable to derive the prey fusion protein with a fixed prey polypeptide rather than a variegated library on the grounds that the single prey fusion protein can be easily tested for its ability to associate with a localized bait protein. In certain embodiments, a variegated bait polypeptide library can be used to create a library of bait fusion proteins to be tested for interaction with a particular prey protein.

In another aspect of the present invention, the DNA sequence encoding the prey protein (or alternatively the bait protein) is embedded in a DNA sequence encoding a conformation-constraining protein (i.e., a protein that decreases the flexibility of the amino and carboxy termini of the prey protein). Such embodiments are preferred where the prey polypeptide is a relatively short peptide, e.g., 5-25 amino acid residues. In general, conformation-constraining proteins act as scaffolds or platforms, which limit the number of possible three dimensional configurations the peptide or protein of interest is free to adopt. Preferred examples of conformation-constraining proteins are thioredoxin or other thioredoxin-like sequences, but many other proteins are also useful for this purpose. Preferably, conformation-constraining proteins are small in size (generally, less than or equal to 200 amino acids), rigid in structure, of known three dimensional configuration, and are able to accommodate insertions of proteins of interest without undue disruption of their structures. A key feature of such proteins is the availability, on their solvent exposed surfaces, of locations where peptide insertions can be made (e.g., the thioredoxin active-site loop).

The subject assay can also be used to generate antibody equivalents for specific determinants, e.g., such as single chain antibodies, minibodies or the like. Indeed, the subject method can be used to identify a novel binding partner for a given epitope/determinant where the new binding partner is a completely artificial polypeptide. For example, a target polypeptide (or epitope thereof) for which an antibody or antibody equivalent is sought can be displayed on either the bait or prey fusion protein. A library of potential binding partners can be arrayed on the other fusion protein, as appropriate. Interactions between the target polypeptide and members of the library of binding partners can be detected according to methods described herein. Thus, the present invention provides a convenient method for identifying recombinant nucleic acid sequences that encode proteins useful in the replacement of, e.g., monoclonal antibodies.

The prey protein is fused in flame to a detection sequence. The detection sequence can be any sequence which permits detection of the specific location of the prey protein within the cell. Preferably, the detection sequence permits detection of the location of the prey protein without further treatment of the cell (e.g., without needing to add additional reagents such as substrates, antibodies, etc.). Additionally, it is preferred that the detection protein be compatible with manual or automatic imaging techniques, such as FACS or fluorescence microscopy. The detection sequence preferably allows the automatic selection of one or more cells containing a positive bait/prey interaction from a large population of cells. Preferred examples of detection sequences include, but are not limited to green fluorescent protein (GFP) (Prasher, D. C. et al., Gene 111, 229-233 (1992) and U.S. Pat. No. 5,625,048); the S26T/V163A mutant of green fluorescent protein (Shibasaki F, et al., Nature 382: 370-373 (1996)); blue fluorescent protein (BFP) (Karatani, et al., Photochem. Photobiol. 55(2):293-299 (1992); Lee, et al., Methods Enzymol. (Biolumin. Chemilumin.) 57:226-234 (1978); and Gast, et al. Biochem. Biophys. Res. Commun. 80(1):14-21 (1978), firefly or bacterial luciferase, or other such fluorescent and luminescent proteins. Alternatively, the detection sequence may encode for a protein such as beta-lactamase or beta-galactosidease that will produce a fluorescent signal upon exposure to a fluorogenic substrate (Tsien et al., U.S. Pat. No. 6,031,094 and Saalnuller A and Mettenleiter T C, J Virol Methods, 44: 99-108 (1993); Lorincz M., et al., Cytometry, 24: 321-9 (1996)). In another embodiment, the detection sequence can encode for an epitope, such as, for example the myc epitope. A labeled antibody, for example, fluorescently labeled anti-myc, can then be used to detect the location of the antigenically-tagged prey protein.

In a preferred embodiment, the prey vector also comprises a selection sequence, although as outlined above, this may not be necessary in some embodiments, for example if the prey vector is a retroviral vector, or if the prey vector is combined with another vector. Preferably, when the bait and prey vectors are distinct, the selection gene of the prey vector is different from the selection gene of the bait vector, to ensure that both vectors are maintained within the cell. However, in some embodiments this may not be required; accordingly, the first and second selection genes may be the same or different.

The selection sequence may also be used as a detection sequence. For example, a prey construct containing a destruction sequence and a selection sequence will become stabilized upon proper localization and binding to a bait construct, whereas improperly localized preys will be degraded. Therefore, cells expressing a bait and prey construct which are capable of interacting would stably express a selection sequence and could be selected based on the growth advantage confered by the selection sequence. In a preferred embodiment a selection sequence is used in conjunction with a detection sequence as means to assay for positive bait-prey interactions. For example, cells expressing prey constructs capable of interacting with a bait may first be isolated based on the growth advantaged conferred by the stable expression of the selection sequence on the prey construct. Cells selected based on growth advantage may then be further assayed for a bait-prey interaction based on determination of the particular subcellular localization of the prey construct in relation to the bait construct. This dual selection/detection method permits increased throughput because the first “selection” round can quickly isolate a population of cells containing a prey construct capable of interacting with the bait without the need to analyze each cell individually. Additonally, the dual selection/detection method helps to reduce the number of false positives since the interaction between the bait and prey constructs is being assayed by two individual methods.

As described above for the bait construct, the prey construct may also contain a rescue sequence. The rescue sequence can facilitate a simple immunoassay for fusion protein expression, e.g. to detect the presence and folding of the fusion protein, or can be used for isolation of the prey construct. The rescue sequence on the prey may be the same as or different from that found on the bait construct.

As described above for the bait construct, the prey construct preferably contains an instability sequence. The instability sequence on the prey vector can significantly enhance the sensitivity of the two-hybrid system of the invention through reduction of background. The instability sequence of the prey construct induces the degradation of most or all of the prey-detection fusion molecules which do not interact with the bait protein and thus do not localize to the desired subcellular location. Therefore, when an instability sequence is included in the prey-detection fusion protein, positive bait/prey interactions may be assayed simply by detecting the presence of the prey construct in the cell. The specificity of the bait prey/interaction may then be confirmed by detecting the specific subcellular localization of the prey-detection fusion protein in relation to the known localization of the bait protein. In the absence of an instability sequence, the pattern of localization of the prey construct must be determined. Detection of the prey construct in a specific pattern that parallels the subcellular localization of the bait protein would be indicative of a positive bait/prey interaction. Generalized or non-specific localization of the prey construct within the cell would be indicative of a lack of interaction between the bait and the prey constructs.

In another preferred embodiment, the prey construct additionally contains a dimerization domain (Table 8). Dimerization of the prey construct will increase its avidity for the bait and thus permit detection of low affinity interactions. A variety of amino acid sequences capable of inducing dimerization are well known to the skilled artisan. Preferred dimerization domains include, but are not limited to, the leucine zipper domain, LexA dimerization domain (Golemis and Brent, Mol. Cell Biol. 12:3006-3014, 1992), a domain comprising the sequence IQRMKQLED KVEELLSKNY HLENEVARLK KLVGER (Blondel et al., Protein Engineering (1991) 4:457-461), a domain comprising the sequence SKVILF (WO 94/28173), the helix-loop-helix region of basic-region helix-loop-helix (“bHLH”) proteins (C. Murre et al., 1989, Cell, 56:777-783). A particularly preferred example of a dimerization domain is that from the yeast GCN4 protein (W. H. Landschultz et al., 1988, Science, 240:1759-1764; A. D. Baxevanis and C. R. Vinson, 1993, Curr. Op. Gen. Devel., 3:278-285; E. K. O'Shea et al., 1989, Science, 243:538-542) (FIG. 15). Preferably, the dimerization domain used to construct the bait fusion is from a species different the host cell so as to decrease the chances that the prey construct will interact with an endogenous protein.

TABLE 8
Exemplary proteins containing a sequence capable of conferring dimerization.

| | | | |
|---|---|---|---|
| Protein/consensus | Dimerization Sequence | Reference | SEQ ID NO |

| | | |
|---|---|---|
| leucine zipper | L-x(6)-L-x(6)OL-x(6)-L | Landschulz et al., |
| domain | | Science 240:1759-1764 |
| | | (1988); Busch et al., |
| | | Trends Genet. 6:36-40 |
| | | (1990); and O'Shea et |
| | | al., Science 243:538- |
| | | 542 (1989) |
| LexA | GenBank Accession No. P03033 | Golemis and Brent, Mol. |
| | | Cell Biol. 12:3006-3014, |
| | | 1992 |
| | IQRMKQLEDKVEELLSKNYHLEN | Blondel et al., Protein |
| | EVARLKKLVGER | Engineering (1991) |
| | | 4:457-461 |
| | SKVILF | WO 94/28173 |
| helix-loop-helix | [DENSTAP]-[KR]-[LIVMAGSNT]- | C. Murre et al., 1989, |
| region of basic- | {FYWCPHKR}-[LIVMT]-[LIVM]- | Cell, 56:777-783 |
| region helix- | x(2)-[STAV]-[LIVMSTACKR]-x- | |
| loop-helix | [VMFYH]-[LIVMTA]-{P}-{P}- | |
| (“bHLH”) | ]LIVMRKHQ] | |
| proteins | | |
| GCN4 protein | GenBank Accession No. AAA34640 | W. H. Landschultz et al., |
| | | 1988, Science, |
| | | 240:1759-1764; A. D. |
| | | Baxevanis and C. R. |
| | | Vinson, 1993, Curr. Op. |
| | | Gen. Devel., 3:278-285; |
| | | E. K. O'Shea et al., |
| | | 1989, Science, 243:538- |
| | | 542 |

In other embodiments, the construct encoding the prey (or bait) fusion protein can include a promoter for in vitro translation (e.g., a T7 promoter) of the target polypeptide (Yavuzer et al., Gene 165: 93 (1995)). Such constructs can be used to eliminate subcloning steps necessary to carry out certain validation assays often undertaken after the initial identification of the protein in the two-hybrid system, e.g., to determine if the binding of the two proteins is truly the result of an interaction between the bait and prey polypeptides per se.

As with the bait protein construct, the elements of the prey construct may be fused together in-frame in any order.

5. Vectors

Using the nucleic acids of the present invention which encode a fusion protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the fusion protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the fusion protein; for example, transcriptional and translational regulatory nucleic acid sequences from Bacillus are preferably used to express the fusion protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring, hybrid or synthetic promoters. Hybrid promoters, which combine elements of more than one promoter, are also known in the art, and are useful in the present invention. In a preferred embodiment, the promoters are strong promoters, allowing high expression in cells, particularly mammalian cells, such as the CMV promoter, particularly in combination with a Tet regulatory element.

In general, the vectors of the present invention utilize two different types of promoters. In a preferred embodiment, the promoters on the bait and test vectors are constitutive, and drive the expression of the fusion proteins and selection genes, if applicable, at a high level. However, it is possible to utilize inducible promoters for the fusion constructs and selection genes, if necessary, for example if toxic proteins are used as either the bait or test proteins.

Preferred promoters for driving expression of the fusion constructs, and the selection genes, if applicable, on the bait and test vectors, include, but are not limited to, cytomegloviral promoters (CMV), SV40, SRα (Takebe et al., Mole. Cell. Biol. 8:466 (1988)), respiratory synsitial viral promoters (RSV), thymine kinase (TK), beta-globin, etc. Particularly preferred promoters are CMV promoters.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

As for all of the vectors described herein, the vector may be extrachromosomal, or may be integrated into the genome of the host cell. In a preferred embodiment, one or more of the vectors may contain a RNA splicing sequence upstream or downstream of the test or bait protein gene to increase the level of gene expression. See Barret et al., Nucleic Acids Res. 1991; Groos et al., Mole. Cell. Biol. 1987; and Budiman et al., Mole. Cell. Biol. 1988.

In addition, in a preferred embodiment, the expression vector contains a selectable marker gene to allow the selection of transformed host cells. Selection genes are well known in the art and will vary with the host cell used.

In a preferred embodiment, either or one or both of the fusion constructs may contain a “rescue” sequence. A rescue sequence is a sequence (either nucleic acid or amino acid) which may be used to purify or isolate either the test or bait proteins or the nucleic acid encoding them. Thus, for example, protein rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST.

Alternatively, the rescue sequence may be a unique oligonucleotide sequence which serves as a probe target site to allow the quick and easy isolation of the retroviral construct, via PCR, related techniques, or hybridization.

Nucleic acids within the scope of the invention may also contain linker sequences, modified restriction endonuclease sites and other sequences useful for molecular cloning, expression or purification of such recombinant polypeptides.

The candidate nucleic acids are introduced into the cells for screening. “Introduced into” means that the nucleic acids enter the cells in a manner suitable for subsequent expression of the nucleic acid. The method of introduction is largely dictated by the targeted cell type. Exemplary methods include CaPO₄ precipitation, liposome fusion, lipofectin®, electroporation, viral infection, etc. The candidate nucleic acids may stably integrate into the genome of the host cell (for example, with retroviral introduction, outlined below), or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets are preferred.

The fusion proteins of the present invention are produced by culturing a host cell transformed with an expression vector containing nucleic acid encoding a fusion protein, under the appropriate conditions to induce or cause expression of the fusion protein. The conditions appropriate for fusion protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important.

In a preferred embodiment, the fusion proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for the fusion protein into MRNA. A promoter will have a transcription initiating region, which is usually placed proximal to the 5′ end of the coding sequence, and a TATA box, located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ termtinus of the mature MRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei. A particularly preferred method utilizes retroviral infection.

For non-retroviral embodiments, suitable vectors are derived from any number of known vectors, including, but not limited to, pCEP4 (Invitrogen), pCI-NEO (Promega), and pBI-EGFP (Clontech). Basically, any mammalian expression vectors with strong promoters such as CMV can be used to construct test or bait vectors. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems.

In a preferred embodiment, one or more retroviral vectors are used. Currently, the most efficient gene transfer methodologies harness the capacity of engineered viruses, such as retroviruses, to bypass natural cellular barriers to exogenous nucleic acid uptake. The use of recombinant retroviruses was pioneered by Richard Mulligan and David Baltimore with the Psi-2 lines and analogous retrovirus packaging systems, based on NIH 3T3 cells (see Mann et al., Cell 33:153-159 (1993), hereby incorporated by reference). Such helper-defective packaging lines are capable of producing all the necessary trans proteins, gag, pol, and env, that are required for packaging, processing, reverse transcription, and integration of recombinant genomes. Those RNA molecules that have in cis the psi. packaging signal are packaged into maturing virions.

Retroviruses are preferred for a number of reasons. First, their derivation is easy. Second, unlike Adenovirus-mediated gene delivery, expression from retroviruses is long-term (adenoviruses do not integrate). Adeno-associated viruses have limited space for genes and regulatory units and there is some controversy as to their ability to integrate. Retroviruses therefore offer the best current compromise in terms of long-term expression, genomic flexibility, and stable integration, among other features. The main advantage of retroviruses is that their integration into the host genome allows for their stable transmission through cell division. This ensures that in cell types that undergo multiple independent maturation steps, such as hematopoietic cell progression, the retrovirus construct will remain resident and continue to express. In addition, transfection efficiencies can be extremely high, thus obviating the need for selection genes in some cases.

A particularly well suited retroviral transfection system is described in Mann et al., supra: Pear et al., PNAS USA 90(18):8392-6 (1993); Kitamura et al., PNAS USA 92:9146-9150 (1995); Kinsella et al., Human Gene Therapy 7:1405-1413; Hofmann et al., PNAS USA 93:5185-5190; Choate et al., Human Gene Therapy 7:2247 (1996); WO 94/19478; PCT US97/01019, and references cited therein, all of which are incorporated by reference.

Any number of suitable retroviral vectors may be used. Generally, the retroviral vectors may include: selectable marker genes under the control of internal ribosome entry sites (IRES), which allows for bicistronic operons and thus greatly facilitates the selection of cells expressing fusion constructs at uniformly high levels; and promoters driving expression of a second gene, placed in sense or anti-sense relative to the 5′ LTR.

Preferred vectors include a vector based on the murine stem cell virus (MSCV) (see Hawley et al., Gene Therapy 1:136 (1994)) and a modified MFG virus (Rivere et al., Genetics 92:6733 (1995)), and pBABE (see PCT US97/01019, incorporated by reference).

As for the other vectors, the retroviral vectors may include inducible and constitutive promoters. Constitutive promoters are preferred for the bait and test vectors, and include, but are not limited to, CMV, SV40, Sr alpha, RSV, and TK.

In addition, it is possible to configure a retroviral vector to allow expression of bait genes or test genes after integration of a bait or test vector in target cells. For example, Tet-inducible retroviruses can be used to express bait or test genes (Hoffman et al., PNAS USA 93:5185 (1996)). Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, indicating that expression is regulated uniformly within the infected cell population. A similar, related system uses a mutated Tet DNA-binding domain such that it bound DNA in the presence of Tet, and was removed in the absence of Tet. Either of these systems is suitable.

The bait and prey vectors can be introduced simultaneously, or sequentially in any order. The bait and prey constructs may be contained on the same or separate constructs.

The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the bait and/or prey constructs by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

6. Host cells

As will be appreciated by those in the art, the type of host cells used in the present invention can vary widely. Basically, any mammalian cells may be used, with mouse, rat, primate and human cells being particularly preferred, although as will be appreciated by those in the art, modifications of the system by pseudotyping allows all eukaryotic cells to be used, preferably higher eukaryotes. Cell types implicated in a wide variety of disease conditions are particularly useful. Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

In one embodiment, the cells may be additionally genetically engineered, that is, contain exogeneous nucleic acid other than the fusion nucleic acid.

7. Exemplary Uses of the Mammalian Two Hybrid System

In a preferred aspect, the present invention provides a method for detecting unique protein-protein interactions that characterize a population or library of proteins by comparing all detectable protein-protein interactions that occur in a population or library with those interactions that occur in another population or library. Furthermore, the method also enables the identification of inhibitors of such protein-protein interactions.

As outlined herein, the present invention is directed to compositions and methods useful as a mammalian two-hybrid system. The present invention can be used in any number of mammalian cells, is highly stable, and is designed to reduce the background signals frequently found in other systems. The present invention thus provides a robust and versatile system to evaluate protein-protein interactions in a wide variety of mammalian cells under any number of different conditions.

Thus, in the two-hybrid system, a first protein, or “bait protein”, as termed herein, is fused to a targeting domain capable of localizing the bait fusion to a particular subcellular site, and a second protein, or “prey protein”, is fused to detector domain which allows visualization of the subcellular locale of the prey protein. If the bait protein and the test protein bind, i.e. have a specific protein-protein interaction, the prey protein will become localized in the host cell in a similar pattern to that of the bait. If there is little or no interaction, the prey will remain dispersed through out the cell or will have a nonspecific localization pattern (e.g., different from that of the bait).

The mammalian two-hybrid system of the present invention can be used for identifying protein-protein interactions, e.g., for generating protein linkage maps, for identifying therapeutic targets, and/or for general cloning strategies. As described above, the two-hybrid system can be used with a cDNA library to produce a variegated array of bait and/or prey proteins which can be screened for interaction with, for example, a known protein expressed as the corresponding fusion protein in the two-hybrid system. Once primary target molecules have been identified, secondary target molecules may be identified in the same manner, using the primary target as the “bait”. In this manner, signalling pathways may be elucidated. Similarly, bioactive peptides specific for secondary target molecules may also be discovered, to allow a number of bioactive peptides to act on a single pathway, for example for combination therapies.

In other embodiments, both the bait and prey proteins can be derived to each provide variegated libraries of polypeptide sequences. One or both libraries can be generated by random or semi-random mutagenesis. For example, random libraries of polypeptide sequences can be “crossed” with one another by simultaneous expression in the subject assay. Such embodiments can be used to identify novel binding pairs of polypeptides.

For example, protein-protein interactions can be detected, and the interacting pairs of proteins isolated and identified, between two populations of proteins wherein both of the populations have a complexity of at least 10 (i.e., both populations contain more than ten distinct proteins). The populations are expressed as bait and prey fusion according to the methods of the invention. In various specific embodiments, one or both of the populations of proteins has a complexity of at least 50, 100, 500, 1,000, 5,000, 10,000, or 50,000; or has a complexity in the range of 25 to 100,000, 100 to 100,000, 50,000 to 100,000, or 10,000 to 500,000. For example, one or both populations can be mammalian cDNA populations, generally having a complexity in the range of 50,000 to 100,000. In a specific embodiment, the invention is capable of detecting substantially all detectable interactions that occur between the component proteins of two populations, each population having a complexity of at least 50, 100, 500, 1000, 5000, 10,000 or 50,000. In a specific embodiment, the two populations are samples (aliquots) of at least 100 or 1000 members of a larger population having a complexity of at least 100, 1000, 5,000, 10,000, or 50,000; in a particular embodiment, the sample is uncharacterized in that the particular identities of all or most of its member proteins are not known.

The populations can be the same or different populations. If it is desired to detect interactions between proteins encoded by a particular DNA population, both protein populations are expressed from chimeric genes comprising DNA sequences representative of that particular DNA population. In another embodiment, one protein population is expressed from chimeric genes comprising cDNA sequences of diseased human tissue, and the other protein population is expressed from chimeric genes comprising cDNA sequences of non-diseased human tissue. In a specific embodiment, one or more of the populations can be uncharacterized in that the identities of all or most of the members of the population are not known. Preferably, the populations are proteins encoded by DNA, e.g., cDNA or genomic DNA or synthetically generated DNA. For example, the populations can be expressed from chimeric genes comprising cDNA sequences from an uncharacterized sample of a population of cDNA from mammalian RNA. Preferably, a cDNA library is used. The cDNA can be, e.g., a normalized or subtracted cDNA population. The cDNA of one or both populations can be cDNA of total mRNA or polyA⁺ RNA or a subset thereof from a particular species, particular cell type, particular age of individual, particular tissue type, disease state or disorder or stage thereof, or stage of development. Accordingly, the invention provides methods of identifying and isolating interacting proteins that are present in or specific to particular species, cell type, age, tissue type, disease state, or disease stage, and also provides methods for comparing the protein-protein interactions present in such particular species, cell type, age, tissue type, disease state, or disease stage (by e.g., using a cDNA library of total mRNA particular to such species, cell type, age, tissue type, disease state, or disease stage, respectively, as both the populations between which interactions are detected) with the protein-protein interactions present in a different species, cell type, age, tissue type, non-diseased state or a different disease stage, or different state of development, respectively. For example, in one embodiment, interactions are detected between identical populations of proteins in which the population of proteins is from cDNA of cancerous or precancerous (e.g., hyperplastic, metaplastic, or dysplastic cells), e.g., of prostate cancer, breast cancer, stomach cancer, lung cancer, ovarian cancer, uterine cancer, etc.; these interactants are then compared to interacting proteins detected between two other identical populations of proteins in which the population of proteins is from cDNA of cells not having the cancer or precancerous condition, as the case may be. In a specific embodiment, cDNA may be obtained from a preexisting cDNA sample or may be prepared from a tissue sample. When cDNA is prepared from tissue samples, methods commonly known in the art can be used. For example, these can consist of largely conventional steps of RNA preparation from the tissue sample, preferably total poly(A) purified RNA is used but less preferably total cellular RNA can be used, RNase extraction, DNase treatment, mRNA purification, and first and second strand cDNA synthesis.

Preferably, the populations of proteins between which interactions are detected are provided by recombinant expression of nucleic acid populations (e.g., cDNA or genomic libraries). Also preferably, the interactions occur intracellularly. In another specific embodiment, recombinant biological libraries expressing random peptides can be used as the source nucleic acid for one or both of the nucleic acid populations.

Alternatively, the subject two hybrid system can be used to map residues of a protein involved in a known protein-protein interaction. Thus, for example, various forms of mutagenesis can be utilized to generate a combinatorial library of either bait or prey polypeptides, and the ability of the corresponding fusion protein to function in the two hybrid system can be assayed. Mutations which result in diminished (or potentiated) binding between the bait and prey fusion proteins can be detected by the level of reporter gene activity. For example, mutants of a particular protein which alter interaction of that protein with another protein can be generated and isolated from a library created, for example, by alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of a protein, e.g., to establish binding domains.

In other embodiments, the two hybrid system can be designed for the isolation of genes encoding proteins which physically interact with a protein/drug complex. The method relies on detecting the reconstitution of a transcriptional activator in the presence of the drug. If the bait and prey fusion proteins are able to interact in a drug-dependent manner, the interaction may be detected by reporter gene expression.

The two hybrid system of the invention can also be used in a three hybrid mode to identify protein targets which bind to particular compound or to screen a library of compounds for their ability to bind to a particular protein target (see Liu et al., U.S. Pat. No. 5,928,868, incorporated herein by reference). To utilize the three hybrid system, the targeting domain of the bait construct is fused to receptor for a known ligand. The ligand is then covalently attached to a compound of interest and is added to the host cell. The ligand-compound fusion becomes immobilized at the subcellular locale of the bait protein through interaction of the receptor with its ligand. The immobilized compound may then be screened against a library of prey proteins to find those which are capable of interacting with the compound. Alternatively, a given ligand may fused to a library of test compounds which are then immobilized to the bait as described. The library of compounds can then be screened for their interaction with one or more bait proteins.

Another aspect of the present invention relates to the use of the mammalian two hybrid system in the development of assays which can be used to screen for drugs which are either agonists or antagonists of a protein-protein interaction of therapeutic consequence. In a general sense, the assay evaluates the ability of a compound to modulate binding between the bait and prey polypeptides. Exemplary compounds which can be screened include peptides, nucleic acids, carbohydrates, small organic molecules, and natural product extract libraries, such as isolated from animals, plants, fungus and/or microbes.

In many drug screening programs which test libraries of compounds and natural extracts, high throughput assays are desirable in order to maximize the number of compounds surveyed in a given period of time. The subject two hybrid system-derived screening assays can be carried out in such a format, and accordingly may be used as a “primary” screen. Accordingly, in an exemplary screening assay of the present invention, a two hybrid system is generated to include specific bait and prey fusion proteins known to interact, and compound(s) of interest. A change in the ability of the prey protein to localize to the bait protein would indicate that a given compound had the ability to affect the specific bait/prey interaction.

In another exemplary embodiment, a therapeutic target devised as the bait-prey complex is contacted with a peptide library with the goal of identifying peptides which potentiate or inhibit the bait-prey interaction. Many techniques are known in the art for expression peptide libraries intracellularly. In one embodiment, the peptide library is provided as part of a chimeric thioredoxin protein, e.g., expressed as part of the active loop (supra).

In yet another embodiment, the mammalian two hybrid system can be generated in the form of a diagnostic assay to detect the interaction of two proteins, e.g., e.g., where the gene from one is isolated from a biopsied cell. For instance, there are many instances where it is desirable to detect mutants which, while expressed at appreciable levels in the cell, are defective at binding other cellular proteins. Such mutants may arise, for example, from fine mutations, e.g., point mutants, which may be impractical to detect by the diagnostic DNA sequencing techniques or by the immunoassays. The present invention accordingly further contemplates diagnostic screening assays which generally comprise cloning one or more cDNAs from a sample of cells, and expressing the cloned gene(s) as part of an two hybrid system under conditions which permit detection of an interaction between that recombinant gene product and a target protein. Accordingly, the present invention provides a convenient method for diagnostically detecting mutations to genes encoding proteins which are unable to physically interact with a target “bait” protein, which method relies on detecting the subcellular localization of the prey construct in relation to the bait construct.

To illustrate, the subject two hybrid system can be used to detect inactivating mutations of the CDK4/p16^{INK4a} interaction. Recent discoveries have brought several cell-cycle regulators into sharp focus as factors in human cancer. Among the most conspicuous types of molecule to emerge from ongoing studies in this field are the cyclin-dependent kinase inhibitors such as p16. (Serrano et al. (1993) Nature 366:704; and Okamoto et al. (1994) PNAS 91:11045) The p16 protein has several hallmarks of a tumor suppressors and is perfectly positioned to regulate critical decisions in cell growth. The p16 gene appears to be a particularly significant target for mutation in sporadic tumors and in at least one form of hereditary cancer. In an exemplary embodiment of the diagnostic two hybrid system, a first hybrid gene comprises the coding sequence for a DNA-binding domain fused in frame to the coding sequence for a bait protein, e.g., CDK4 or CDK6. The second hybrid protein encodes a polymerase interaction domain fused in frame to a gene encoding the sample protein, e.g. a p16 gene (cDNA) amplified from a cell sample of a patient. If the bait and sample proteins are able to interact, e.g., form a CDK/p16 complex, then RNA polymerase is recruited to the promoter of a reporter gene which is operably linked to a DBD recognition element, thereby causing expression of the reporter gene.

Moreover, it will be apparent that the subject two hybrid assay can be used generally to detect mutations in other cellular proteins which disrupt protein-protein interactions. For example, it has been shown that the transcription factor E2F-4 is bound to the p130 pocket protein, and that such binding effectively suppresses E2F-4-mediated trans-activation required for control of G₀/G₁ transition. Mutants which result in disruption of this interaction can be detected in the subject assay.

Similarly, Rb and Rb-like proteins (such as p107) act to control cell-cycle progression through the formation of complexes with several cellular proteins. In fact, a recent article concerning familial retinoblastoma has reported a new class of Rb mutants found in retinal lesions, which mutants were defective in protein binding (“pocket”) activity (see, for example, Kratzke et al. (1994) Oncogene 9:1321-1326). Moreover, mutant forms of c-myc have been demonstrated in various lymphomas, e.g., Burkitt lymphomas, which mutants are resistant to p107-mediated suppression. Accordingly, the diagnostic two hybrid assay of the present invention can be used to detect mutations in Rb or Rb-like proteins which disrupt binding to other cellular proteins, e.g., myc, E2F, c-Ab1, or upstream binding factor (UBF), or vice-versa.

In another embodiment, the subject diagnostic assay can be employed to detect mutations which disrupt binding of the p53 protein with other cellular proteins, as for example, the Wilm's tumor suppresser protein WT1. Recent observations by Maheswaran et al. (1993, PNAS 90:5100-5104) have demonstrated that p53 can physically interact with WT1, and that this interaction modulates the ability of each protein to transactivate their respective targets. In fact, in contrast to the proposed function of WT1 as a transcriptional repressor, potent transcriptional activation by WT1 of reporter genes driven by EGR1 in cells lacking wild type p53 indicates that transcriptional repression is not an intrinsic property of WT1. Instead, transcriptional repression by WT1 may result from its interaction with p53. Accordingly, mutations in p53 which do not effect the cellular concentration of this protein, but which rather down regulate its ability to bind to and repress WT1, may give rise to Wilm' tumors, and other disease states associated with deregulation of WT1.

In still another embodiment, the diagnostic two hybrid assay can be used to detect mutations in pairs of signal transduction proteins. For example, the present assay can be used to detect mutations in the ras protein or other cellular proteins which interact with ras, e.g., ras GTPase activating proteins (GAPs).

The method of the present invention, as described above, may be practiced using a kit for detecting interaction between a target protein and a sample protein. In an illustrative embodiment, the kit includes two vectors, a host cell, and (optionally) a set of primers for cloning one or more target proteins from a patient sample. The first vector may contain a promoter, a transcription termination signal, and other transcription and translation signals functionally associated with the first chimeric gene in order to direct the expression of the first chimeric gene. The first chimeric gene includes a DNA sequence that encodes a targeting domain and a unique restriction site(s) for inserting a DNA sequence encoding the bait protein or protein fragment in such a manner that the bait protein is expressed as part of a hybrid protein with the targeting domain. The first vector also includes a means for replicating itself (e.g., an origin of replication) in the host cell. In preferred embodiments, the first vector also includes a first marker gene, the expression of which in the host cell permits selection of cells containing the first marker gene from cells that do not contain the first marker gene.

The kit also includes a second vector which contains a second chimeric gene. The second chimeric gene also includes a promoter and other relevant transcription and translation sequences to direct expression of the prey fusion protein. The second chimeric gene also includes a DNA sequence that encodes a detection sequence and a unique restriction site(s) to insert a DNA sequence encoding the sample protein, or fragment thereof, into the vector in such a manner that the prey protein is capable of being expressed as part of a hybrid protein with the detection sequence.

In general, the kit will also be provided with one of the two vectors already including the bait protein. For example, the kit can be configured for detecting mutations to a p16-gene which result in loss of binding to CDK4. Accordingly, the first vector could be provided with a CDK4 open reading frame fused in frame to the DNA-binding domain to provide a CDK4 bait protein. p16-gene open reading frames can be cloned from a cell sample and ligated into the second vector in frame with the detection sequence.

Where the kit also provides primers for cloning a prey gene into the two hybrid assay vectors, the primers will preferably include restriction endonuclease sites for facilitating ligation of the amplified gene into the insertion site flanking the targeting domain or detection sequence.

Accordingly in using the kit, the interaction of the bait protein and the prey protein in the host cell can be determined by detecting the subcellular localization of the prey protein in relation to the know subcellular locale of the bait construct. The cells containing the two hybrid proteins are incubated in/on an appropriate medium and the cells are monitored for the expression and/or localization of the prey fusion. A positive test for this activity is an indication that the target protein and the sample protein have interacted. Such interaction causes the prey protein to become localized in the host cell in a pattern similar to that of the bait.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Localization of Kinetochore Proteins at Discrete Subcellular Sites

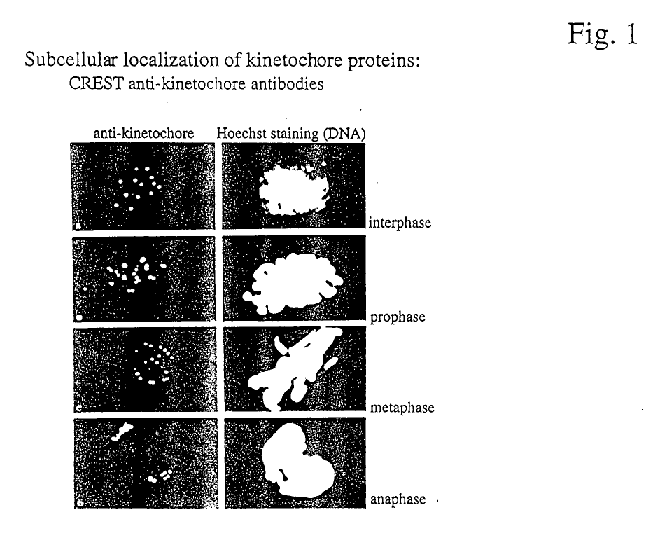

The kinetochore is a multiprotein complex which assembles at the centromere of chromosomes. Kinetochore interactions with spindle microtubules during mitosis play critical roles in the alignment of chromosomes and the segregation of sister chromatids. Patients with the calcinosis/Raynaud's phenomenon/esophageal dysmotility/sclerodactyly/telangiectasia variant (or CREST syndrome) of scleroderma produce autoantibodies to kinetochore proteins and that these antigens were present on both interphase and mitotic chromosomes (Moroi Y, et al., Proc Natl Acad Sci USA, 77:1627-31 (1980)). These antibodies have been employed to identify cDNAs encoding several of the kinetochore antigens, including CENP-A, CENP-13, and CENP-C (Moroi Y, et al., Proc Natl Acad Sci USA, 77:1627-31 (1980); Maney T, et al., Int Rev Cytol, 194: 67-131 (2000)). As seen in FIG. 1, the pattern of kinetochore labeling is discrete, uniform, and intense.

FIG. 1 shows the kinetochore immunofluorescence pattern in Chinese hamster ovary (CHO) cells stained with autoantibodies from patients with the CREST syndrome of scleroderma. Left panels, Indirect immunofluorescence of CREST antibodies. Right panels, Hoechst dye staining showing chromatin pattern. A panels, interphase pattern of single kinetochores; B panels, prophase showing duplicated kinetochores on sister chromatids; C panels, metaphase cells showing kinetochore alignment; D panels, anaphase showing segregation of sister kinetochores to opposite spindle poles.

### Example 2

### Targeting of Myc-Tagged Human CENP-C to Kinetochores

CENP-C possesses several important characteristics which render it an ideal platform for the two-hybrid system in animal cells described herein. Exogenous human CENP-C can be expressed in cells using standard transfection techniques and this “tagged”-CENP-C will label each kinetochore in the cells (Lanini L and McKeon F, Mol Biol Cell, 6:1049-1059 (1995)). Thus the information for targeting and assembly of CENP-C at the kinetochore is completely contained within the CENP-C coding sequence.

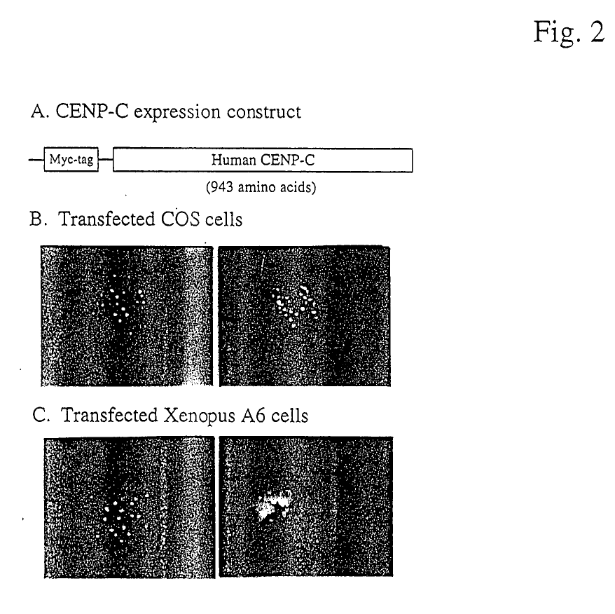

FIG. 2 shows the targeting of myc-tagged human CENP-C to kinetochores. Panel A shows the expression construct used in these experiments. Panel B shows the expression of human myc-tagged CENP-C in COS (African green monkey) cells with targeting to kinetochores in both interphase (left panel) and metaphase (right panel). Anti-Myc staining is shown in red, while DNA staining with Hoeschst dye is shown in blue. Panel C shows human CENP-C targeting to kinetochores of nonprimate cells. Expression of human CENP-C in Xenopus A6 cells with similar interphase (left) and metaphase (right) patterns of kinetochore localization as seen in primate cells.

Human CENP-C targets to the kinetochores of a wide variety of cells in addition to those of human origin, including those of mouse, hamster, and frogs (FIG. 2). Thus bait fusions of human CENP-C constructs could be utilized in a wide variety of animal cells. Additionally, similar concepts could be applied to other species, including flies (Drosophila) and worms (C. elegans).

### Example 3

### Targeting of CENP-C-Beta-Galactosidase Fusion to the Kinetochore

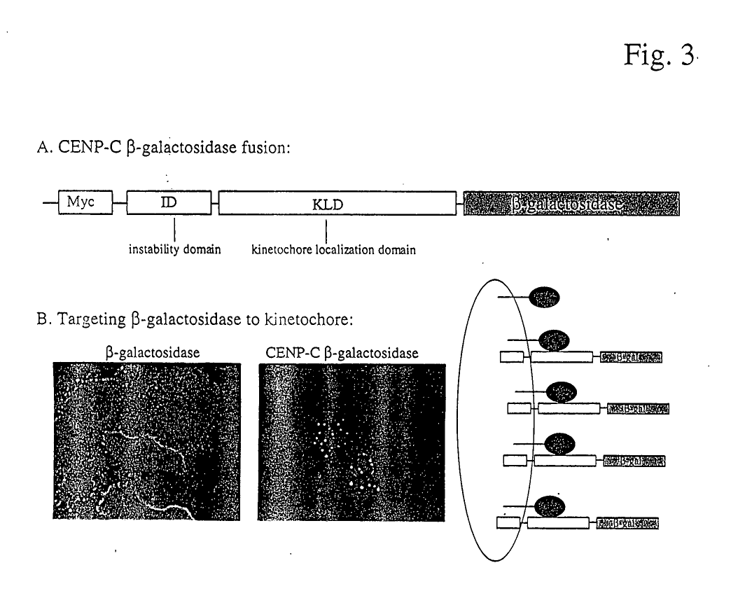

Fusions between CENP-C and unrelated proteins, such as beta-galactosidase, can be expressed in cells and ectopically target the fused protein to the kinetochore (FIG. 3). Therefore, the patterning of the kinetochores, a numerically and spacially precise array, can be used to assay for interacting proteins which assume the identical pattern. The kinetochore pattern is a numerically defined entity in the cell and therefore easily “scoreable” visually or by using automated detection systems. Similar two-hybrid screens could be developed by linking the “bait” proteins to any protein that has a particular subcellular localization.

FIG. 3 shows the targeting of CENP-C-beta-galactosidase to the kinetochore. Panel A shows the expression construct used in these experiments. The construct contains CENP-C coding sequences, including the instability domain (ID) and the kintochore localization domain (KLD), fused in-frame to the beta-galactosidase coding sequences. Panel B shows that unfused beta-galactosidase transfected into BHK cells is expressed at very high levels and localizes to both the cytoplasm and the nucleus (left panel). In contrast, the CENP-C-beta-galactosidase fusion protein expressed in BHK cells accumulates at very low levels and localizes to the kinetochore (right panel). The schematic to the right shows the docking of the CENP-C-beta-galactosidase fusion protein to the kinetochore.

### Example 4

### Targeting Dependent Stability of CENP-C

The 46 kinetochores of a human in total represent an exceedingly small surface area for targeting (0.5 pm²; approximately 1:1000 that of the typical cell surface) and yet contains a high concentration of CENP-C binding sites. Therefore, proteins (the “prey”) interacting with the bait portion of the CENP-C fusion protein would also be targeted into the same highly focal kinetochore sites, yielding readily detectible point source patterns.

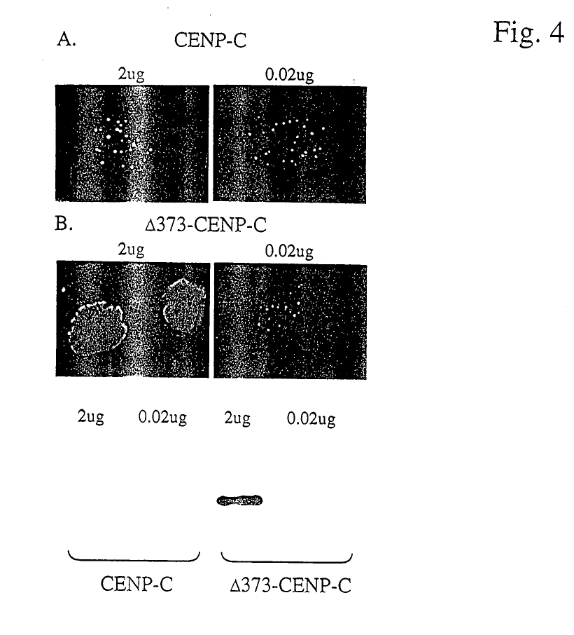

Additionally, overexpression of CENP-C does not lead to higher kinetochore labeling or to an increase in mistargeted CENP-C at other subcellular locations (Lanini L and McKeon F, Mol Biol Cell, 6: 1049-1059 (1995)). Therefore, expression of CENP-C fusion proteins at imprecise levels still provides precise kinetochore labeling. This phenomenon appears to be dependent on the instability domain of CENP-C (Lanini L and McKeon F, Mol Biol Cell, 6: 1049-1059 (1995)), as its removal renders CENP-C stable despite mistargeting (FIG. 4).

FIG. 4 shows the targeting-dependent stability of CENP-C. Panel A, left, shows the kinetochore labeling in BHK cells on a coverslip transfected with 2 μg of a CENP-C expression plasmid. Panel A, right, shows the kinetochore labeling in BHK cells on a coverslip transfected with 0.02 μg of a CENP-C expression plasmid. Panel B, left, shows the labeling pattern in BHK cells on a coverslip transfected with 2 μg of a Δ373-CENP-C expression plasmid (i.e., lacking the instability domain). Panel B, right, shows the kinetochore labeling in BHK cells on a coverslip transfected with 0.02 μg Δ373-CENP-C expression plasmid. Panel C is a Western blot of cells corresponding to Panels A and B showing a marked overexpression of CENP-C lacking the first 373 amino acids, corresponding to the instability domain.

### Example 5

### Destabilization of Beta-Galactosidase by the CENP-C Destruction Box

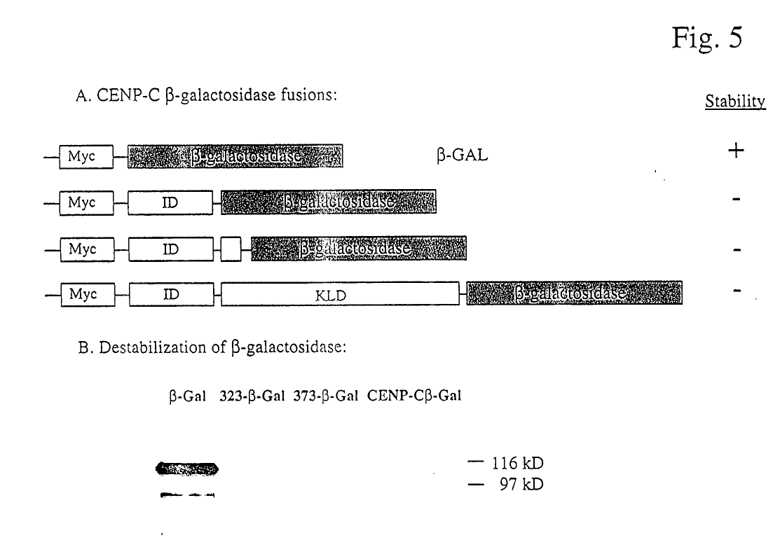

Significantly, the CENP-C instability domain can be transferred to other proteins, such as beta-galactosidase, and confer similar destabilizing effects on the protein to which it is fused (FIG. 5). The basis for this phenomenon appears to be a domain within the CENP-C protein that promotes the destruction of CENP-C molecules that fail to assemble at the kinetochores. How this N-terminal domain destabilizes mistargeted CENP-C is unclear, but its destructive potential is apparently muted by protein-protein interactions at the kinetochore. Destabilization of prey proteins that fail to target to a particular subcellular localization is a preferred embodiment of this invention as it will allow whole cell preliminary screenings of interactions, for example, using FACS analysis.

FIG. 5 shows the destabilization of beta-galactosidase by the CENP-C destruction box. Panel A is a schematic of the fusion proteins used in these experiments which contain all of the beta-galactosidase coding region and portions (N-terminal 323 and 373 aa) or all of the human CENP-C protein. Panel B is a Western blot for beta-galactosidase protein upon transfection of the constructs shown in Panel A into BHK cells. Only the wildtype beta-galactosidase is detected, whereas the 323-, 373-, and full-length CENP-C-beta-galactosidase fusion proteins are not detectable.

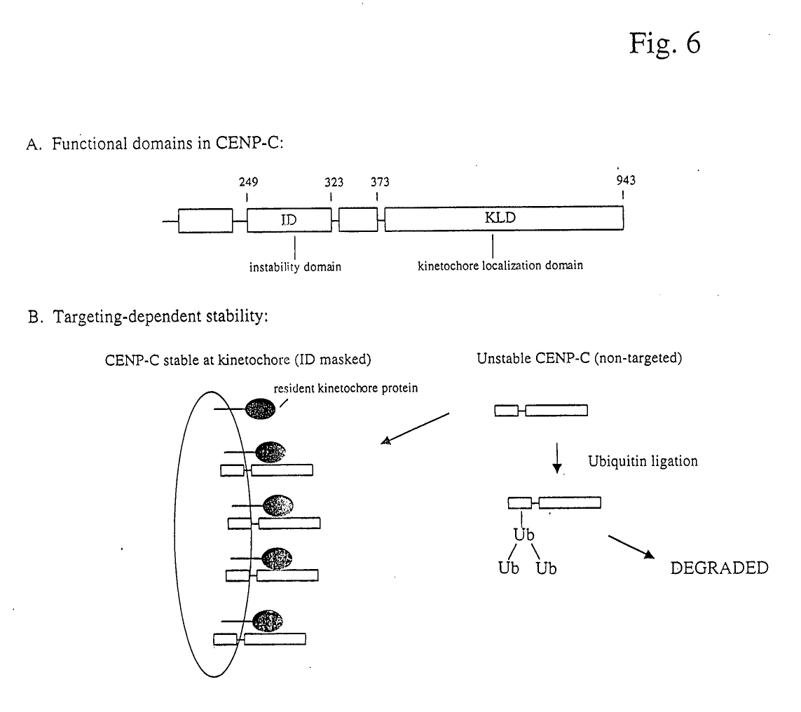

FIG. 6 is a schematic of the CENP-C instability domain function. Panel A shows the domains in CENP-C, including the instability domain (ID), from amino acids 249-323, and the kinetochore localization domain (KLD), from amino acids 373-943. Panel B shows a schematic of targeting dependent stability of CENP-C. CENP-C proteins that assemble at the kinetochore effectively “mask” their instability domain and thus are not affected by the ubiquitin ligation enzymes that promote the destruction of non-targeted CENP-C.

### Example 6

### A Single CENP-C Vector is Sufficient to Saturate the Kinetochore

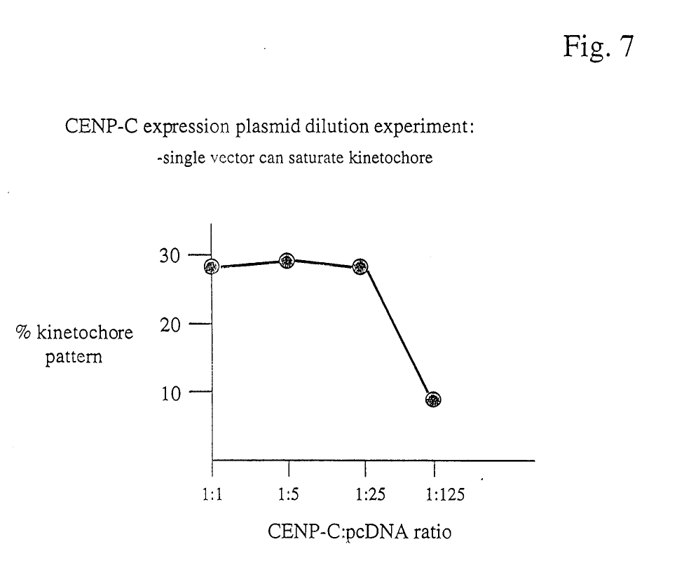

A single CENP-C expression plasmid was found to be sufficient for saturation of the kinetochore. This conclusion was derived form the analysis of cells transfected with varying ratios of CENP-C expression plasmid as compared to empty vector (FIG. 7). These data suggested that in the standard calcium-phosphate mediated transfection, a typical cell acquires somewhere between 30 and 50 plasmids. When all of these plasmids were CENP-C expression vectors, we observed precise and directed labeling of the kinetochores. However, as we diluted the CENP-C expression plasmid with “empty” vector not containing CENP-C coding sequences, we noted essentially identical signals in cells transfected with CENP-C:empty vector ratios of 1:1, 1:5, 1:25, and 1:125 indicating that a single CENP-C expression plasmid was sufficient for saturating the CENP-C binding sites at the kinetochore. Such a finding indicates that the methods of the invention will not be affected by the sensitivity troubles that have been a fundamental problem of the prior art mammalian two-hybrid systems. If one CENP-C expression plasmid can saturate the kinetochore, it is likely that the expression of an interacting protein from a single “prey” vector, appropriately tagged, could yield a detectable kinetochore pattern. Exploitation of the ability of proteins to accumulate at a particular subcellular localization permits highly sensitive investigations of protein-protein interactions in animal cells. This discovery is a significant achievement in the development of mammalian two-hybrid methodology.

FIG. 7 shows that a single CENP-C vector is sufficient to saturate the kinetochore. BHK cells were transfected with constant amounts of DNA consisting of varying ratios of CENP-C expression vector and backbone pcDNA3 vector lacking an insert. Cells were scored for kinetochore signals after staining with the anti-Myc epitope antibody and a secondary Cy3-conjugated anti-mouse IgG antibody, and expressed as a percentage of total cells counted.

### Example 7

### Design of a CENP-C “Bait” Recombinant Retrovirus and Production of a Stable Cell Line

FIG. 8 shows an exemplary design for a “bait” recombinant retrovirus construct and stable cell lines. Panel A is a schematic of a bait construct in a retroviral backbone. Transcription is driven from the promoter in the LTR. An N-terminal truncation of CENP-C that retains the instability sequence (ID) is fused to a Myc epitope tag and then the coding sequence for the bait protein. Panel B is a schematic of the CENP-C-bait docking at the kinetochore. The instability sequence is masked by interaction with kinetochore proteins upon docking. Panel C is a photomicrograph of NS-1 cells showing disposition of CENP-C-bait fusions at the kinetochore visualized using the anti-myc antibody (described above).

### Example 8

### Exemplary Protocol for the Two-Hybrid Method of the Invention

The basic screen starts with a stable cell line expressing the bait fusion protein (e.g., a CENP-C-bait fusion). This line can be made by retroviral transduction or by more traditional transfection procedures using selectable markers on the expression vector.

The sequence of the screening events are:

1. Infection of NS-1 cells with the recombinant retrovirus containing the bait construct;

2. Selection of cells containing bait construct using resistance marker (e.g., G414, hygromycin, etc.);

3. Confirmation of kinetochore localization of bait using anti-myc antibody labeling (as described above);

4. Expansion of selected cells, reconfirmation of presence of bait construct and correct localization of bait protein at kinetochores, and storage of aliquots of bait containing cell line;

5. Infection of an aliquot of bait containing cells (e.g., ˜4×106 cells) with a GFP-prey recombinant retrovirus construct;

6. Optical identification and/or FACS analysis of bait and prey containing cells to obtain single cell cloning of positive interactors (i.e., protein-protein interaction between bait and prey molecule).

### Example 9

### Construction of a Prey Library

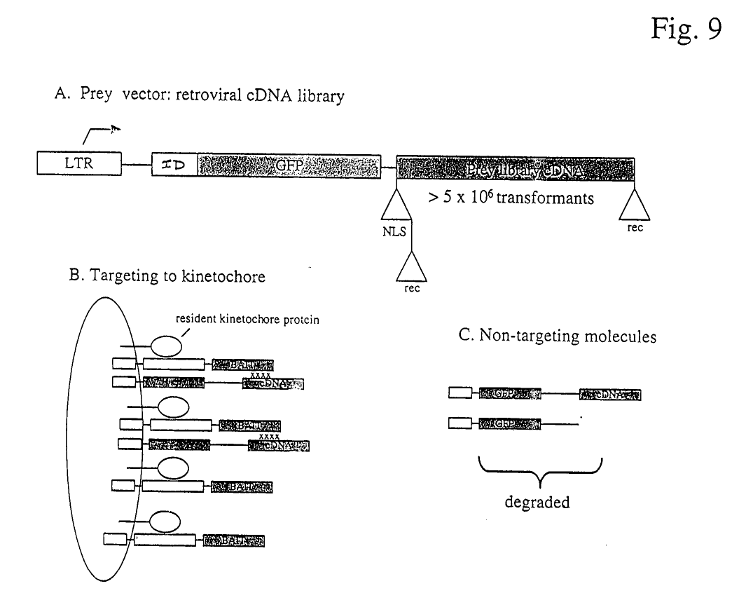

Cells containing baits localized in the unique subcellular pattern of kinetochores (FIG. 8) are then exposed to a library of tagged prey constructs to allow identification of interacting prey proteins. The construction of this retroviral library is in a vector backbone and prey is expressed as a GFP fusion protein. In this case, the GFP portion of the vector utilizes the S26T/N163A double mutant (Shibasaki F, et al., Nature 382: 370-373 (1996)), which possesses a higher quantal yield than native GFP or other GFP mutants, making it an exemplary tag for the prey cDNA libraries of the system. To ensure that the expressed GFP-fusion proteins enter the nucleus, a standard SV40 nuclear localization sequence is inserted into the retroviral vector (FIG. 9). The retroviral transduction system generally introduces a single recombinant virus per cell and thereby converts each cell into a unique assay of a single prey species. The CENP-C instability sequence (ID) is used to suppress the stability of non-interacting prey fusion proteins which fail to target the kinetochore.

FIG. 9 shows a schematic of the prey library construction and of the bait-prey interaction. Panel A shows the backbone of the prey vector which is derived from a retrovirus wherein an LTR drives expression of a hybrid fusion protein. The fusion protein consists of the CENP-C instability sequence (CENP-C aa 240-373), the S26T/N163A GFP double mutant coding sequence, SV40 nuclear location signal sequence, and open reading frames representing cloned cDNA inserts produced by standard cDNA library technologies. Panel B is a schematic showing the docking of prey fusion proteins at the kinetochore by virtue of their interaction with bait molecules docked at that site via CENP-C. Panel C is a schematic showing fusion proteins from this library that would be subject to degradation, including GFP molecules that fail to generate prey fusions (out of frame) and prey fusions that fail to interact with the kinetochore.

### Example 10

### Screening Methods for Identification of Positive Interactors

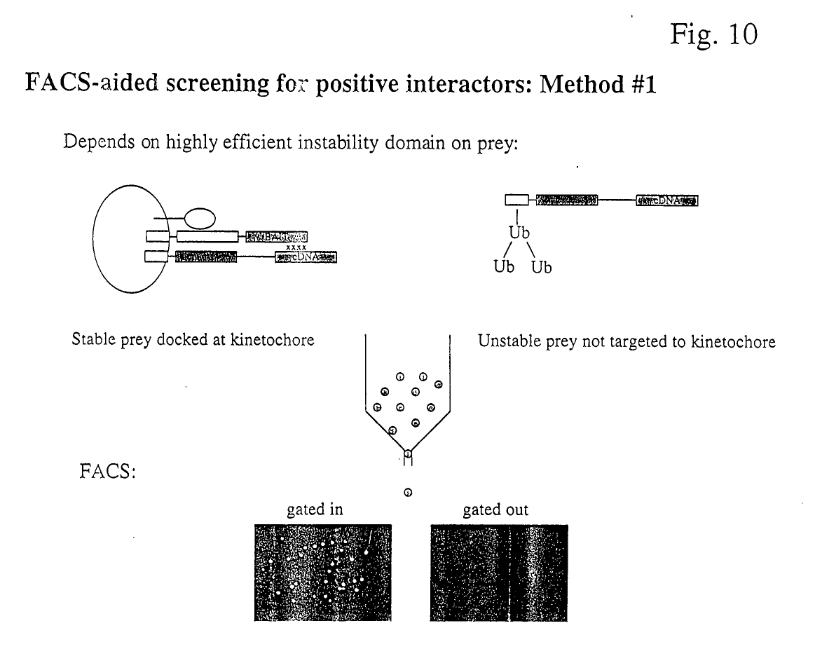

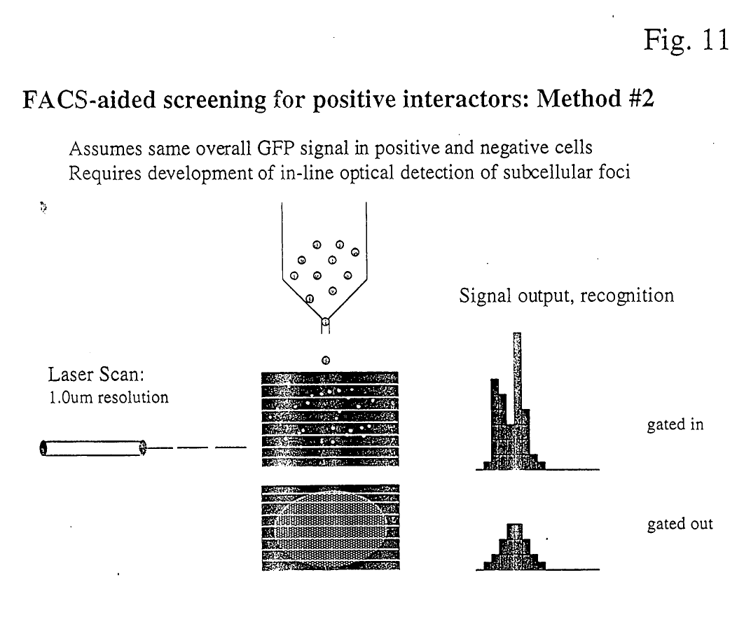

Several methods are envisioned for screening and identification of interacting prey. The methods may be used separately or interactively. Should the CENP-C instability domain efficiently degrade all prey that fail to target to the kinetochore, whole populations of cells infected with the prey library can be prescreened by FACS for those bearing signal (FIG. 10).

FIG. 10 shows a first method for FACS-aided screening for positive interactors. The schematic shows a detection method for protein-protein interactions in mammalian cells wherein the GFP-fused prey is stable when docked at the kinetochore and all non-docked prey are degraded by proteosomes in a ubiquitin- and instability domain-dependent manner. Should the CENP-C instability domain efficiently degrade all prey that fail to target to the kinetochore, whole populations of cells infected with the prey library can be prescreened by FACS for those bearing signal.

As FACS has the capability of assaying 20,000 cells per minute, a typical screen of 3,000,000 cells should take on the order of 2.5 hours. A cell sorter linked to 96 or 384 well plates would accomplish the single cell cloning requirements for expansion of clones for determination of prey identity and subsequent screening for interaction disruptors. It is anticipated that a secondary screen of the putative positives would be accomplished by direct analysis using optical methods. NS-1 cells can be examined under FITC optics for the distinctive kinetochore pattern (manual or programmed). Such cells can be captured for single cell cloning using the backfilling function of a controlled micropipet, and placed into individual wells with appropriate feeder cells. Expanded clones will by analyzed by PCR from the integrated retrovirus using primers specific to the junctions surrounding the cDNA insert. The PCR products will be sequenced and data compared with the available databases. Much of this can be automated and the sequencing outsourced.

A second approach to automating the detection of protein-protein interactions in this system is to assess the particulate nature of prey signal in a cell. Using methods which could determine the signal in multiple sections of a given cell, it should be possible to determine if the prey is diffusely distributed, and therefore not in association with the kinetochore. Alternatively, cells with a kinetochore localization pattern would show signal heterogeneity throughout the cell (FIG. 11). Such a system would preferably be integrated with standard FACS technology.

FIG. 11 shows a second method for FACS-aided screening for positive interactors. The schematic shows a screening method for interactions in mammalian cells in which the majority of cells show uniform nuclear distribution of GFP-tagged prey molecules indicative of non-interactors. In the FACS chamber, a laser emitting light that excites GFP scans an individual cell multiple times and the output is then read to determine either uniform (bell-shaped) or non-uniform (heterogeneous curve) signals. Those with the non-uniform signals are gated in for single cell cloning.

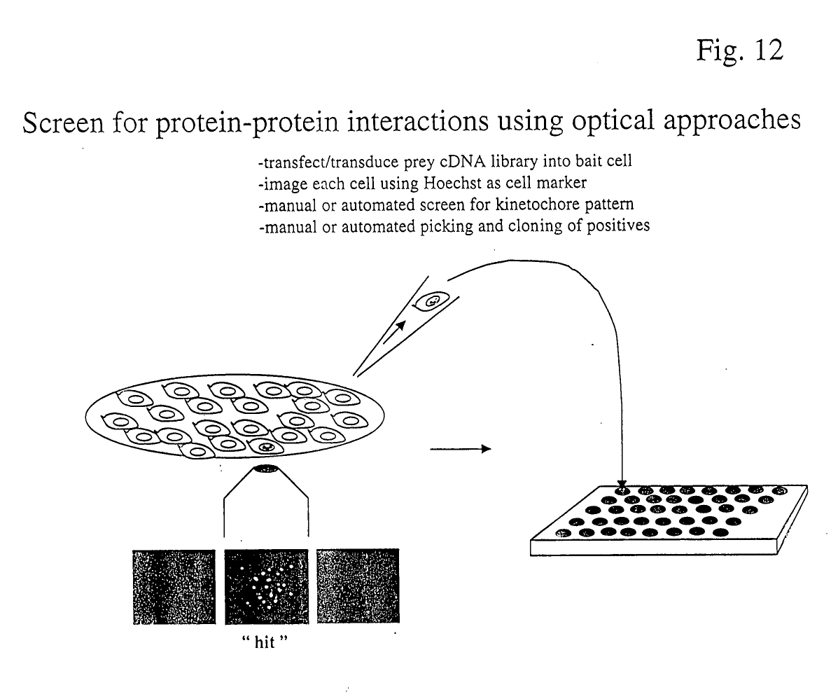

It is anticipated that positive interactions between CENP-C-bait fusions and prey proteins can also be detected using optical imaging either manually or with pattern recognition software. Thus bait-expressing cells could be transduced with the prey cDNA libraries and individual cells with the desired patterns isolated manually or with the aid of software recognition (FIG. 12).

FIG. 12 is a schematic showing imaging and selection of cells with a positive bait/prey interaction. An additional method of identifying and cloning of cells exhibiting interactions is to directly image cells using a standard lens and motorized stage. Cells could be identified using nuclear markers such as Hoechst dye and images processed manually, or by image analysis software, to chose those with kinetochore patterns. Positive cells could then be picked manually, or in an automated fashion, using micropipets such that individual cells could be expanded for prey analysis. Single cell cloning of positive clones yields an immediate assay for screens of small molecules that directly or secondarily affect the protein-protein interaction. Briefly, clones are grown in 384 well plates, drugs introduced robotically, and the patterns assessed by optical screens using established algorithms.

To aid in the screening of whole populations of cells for those with the proper interactions without direct visual screening of kinetochore-prey interactions, a luminescence or fluorescence method would allow FACS isolation of cells. For example, the CENP-C instability domain contained on the prey construct will cause the degradation of prey molecules that fail to localize to the kinetochore. Prey constructs containing beta-lactamase or beta-galactosidase fusions would permit the use of membrane permeant substrates that fluoresce or emit light as a screen for positive interactors. Thus constructs that incorporate beta-lactamase or beta-galactosidase into the ID-CENP-C-GFP-Prey fusion would permit the detection of stable, and presumably kinetochore localized, interactors over those which fail to dock at the kinetochore. Fluorogenic substrates for beta-lactamase are described in Tsien et al., U.S. Pat. No. 6,031,094 and for beta-galactosidase (FACS-Gal, Saalmuller A and Mettenleiter T C, J Virol Methods, 44: 99-108 (1993); Lorincz M., et al., Cytometry, 24: 321-9 (1996)).

### Example 11

### Screening of Small Molecules Against Established Interactions

To find molecules that would directly or indirectly disrupt protein-protein interactions, the cloned cell line demonstrating a given interaction would be expanded, plated into appropriate multiwell dishes, and incubated with a given small molecule. Disruption of the bait/prey interaction will lead to loss of localization of the prey molecule. The assay wells can be screened for the loss of kinetochore association of the prey using automated imaging systems. Alternatively, loss of localization of the prey may lead to its degradation due to the presence of an instability or degradation domain on the prey construct. Degradation of the prey could be detected by luminescence, if a prey-luciferase or prey beta-galactosidase fusion is used in conjunction with appropriate chemiluminescence substrates, or by loss of a selectable marker under the appropriate selective conditions.

### Example 12

### Mammalian Three-hybrid System for Identification of Drug Targets

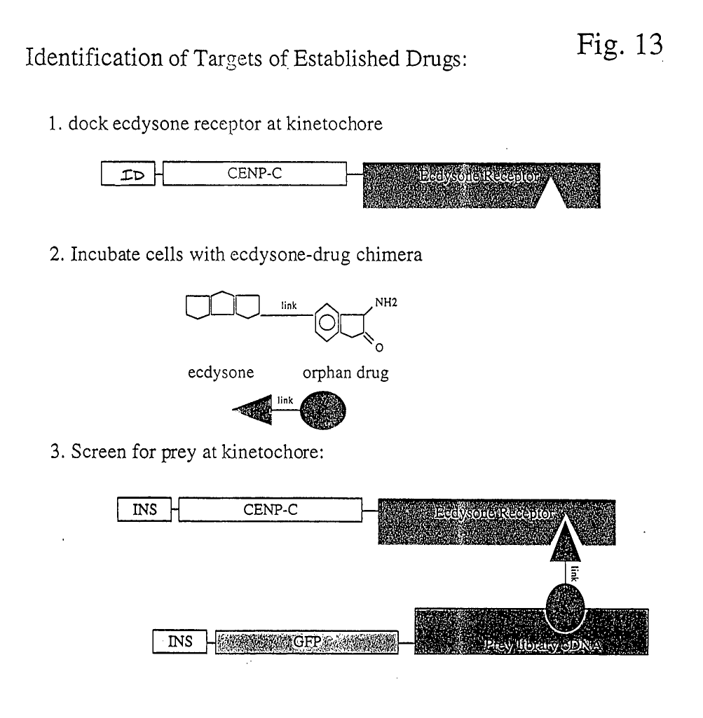

An additional use for CENP-C-mediated localization of proteins to the kinetochore is addressing the problem of drug targets. Many drugs are isolated in biological screens but their specific targets are unknown. Alternatively, drugs isolated on the basis of blocking a specific biochemical process may have additional targets which lead to undesired effects. CENP-C could therefore be used to specifically dock a small molecule or peptide at the kinetochore, and interacting proteins detected by the methods described above (FIG. 13).

FIG. 13 is a schematic of the mammalian “three-hybrid system” to identify drug targets. To screen prey libraries of cDNAs that encode for proteins that interact with a known compound, the compound is targeted to the kinetochore using a presenting receptor fused to CENP-C. In the example presented, the compound is chemically linked to a known ligand such as ecdysone, and the ecdysone receptor is fused to CENP-C. The ecdysone-test compound linkage is then added to cells, and interactions with prey molecules assessed by GFP labeling of kinetochores.

One means of docking the compound in question at the kinetochore is to generate a CENP-C-steroid receptor fusion protein using the model of Liu et al. (Licitra E J and Liu J O, Proc Natl Acad Sci USA, 93: 12817-12821 (1996); Liu et al., U.S. Pat. No. 5,928,868). Examples of such docking receptors are the glucocorticoid receptor, the estrogen receptor, and the ecdysone receptor. The kd's for this class of receptors is on the order of 1-10 nM, and the chemistry of steroids for generating spaced linkages to what would be “bait” compounds is well established. In these examples, prey proteins binding to the kinetochore as a function of the linked moiety would be candidates for the drug target.

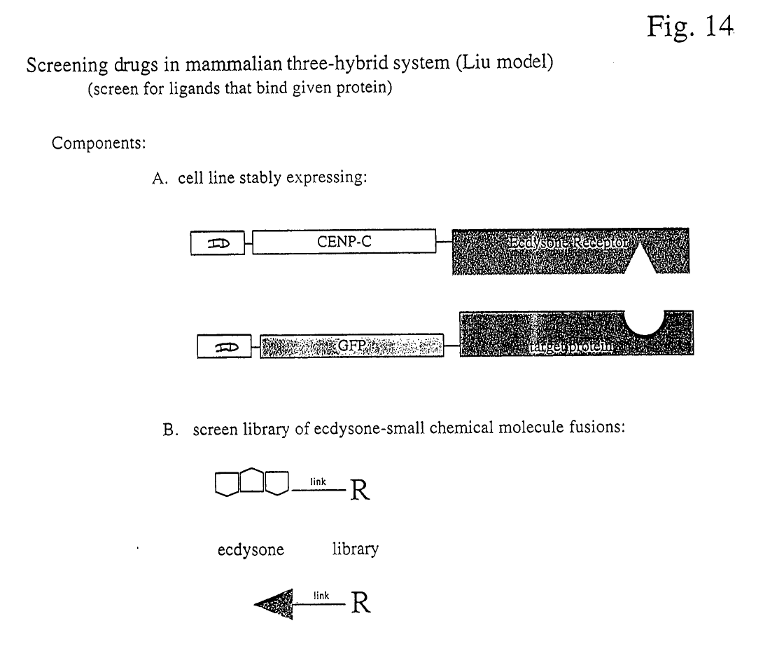

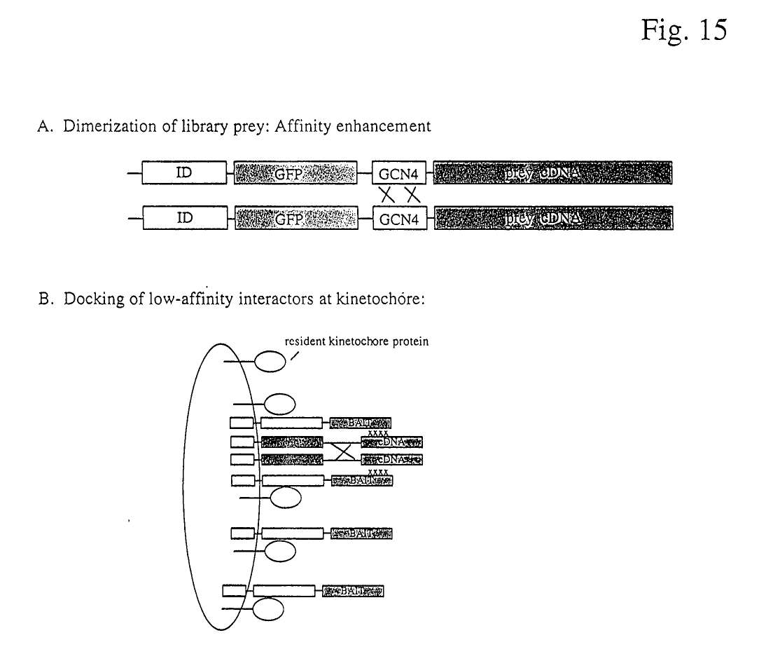

FIG. 14 is a schematic of the components for a mammalian three-hybrid system for drug screening. Panel A shows exemplary constructs for production of a stable cell line expressing kinetochore targeted ecdysone receptor and GFP-tagged target protein. Panel B shows a library of ecdysone-small molecule fusions which may be screened so as to identify candidates capable of localizing the target protein to the kinetochore and thus stabilizing its expression.

One application of the CENP-C based mammalian three-hybrid screen would be to screen for small molecules that interact with a specific protein, essentially the reverse of that described above (FIG. 13). Briefly, a library of ecdysone-small molecule fusions is made through combinatorial chemistry, and screened against a stable cell line expressing the CENP-C-ecdysone receptor and the GFP-target protein fusion (FIG. 14).

### Example 13

### Affinity Enhancement Through Dimerization of the Prey Molecules

One innovation that might allow us to detect “low” affinity interactions is to dimerize the prey (Hu J C, et al., Science, 250:1400-3 (1990)). A dimerized prey will have higher avidity with the bait due to the constrained interactions of each low affinity interaction, which in general terms acts to multiply the affinity constants. For example, the kinetochore may be associated with a multitude of CENP-C-fused bait molecules docked at a high concentration. Therefore if a monomer prey molecule showed even weak affinity for the docked bait, the dimerized, or multimerized, molecule would have a much slower apparent off-rate, and therefore a considerably higher affinity for the bait array. The prey may be dimerized by including any sort of dimerization motif into the prey molecule. Although numerous small dimerization motifs exist, that of the yeast GCN4 protein has been used extensively and could be incorporated into the prey molecule (FIG. 15). Use of a motif from a species different from the host cell used for the testing may decrease the chances of an interaction between the prey construct and an endogenous protein of the host cell.

FIG. 15 shows a schematic for use of a dimerization domain to enhance detection of bait/prey interactions. In this example, the GCN4 dimerization motif is used in the prey construct to enhance the detection of weak interactions with bait at the kinetochore.

### Example 14

### Positive Selection for Protein-Protein Interactions

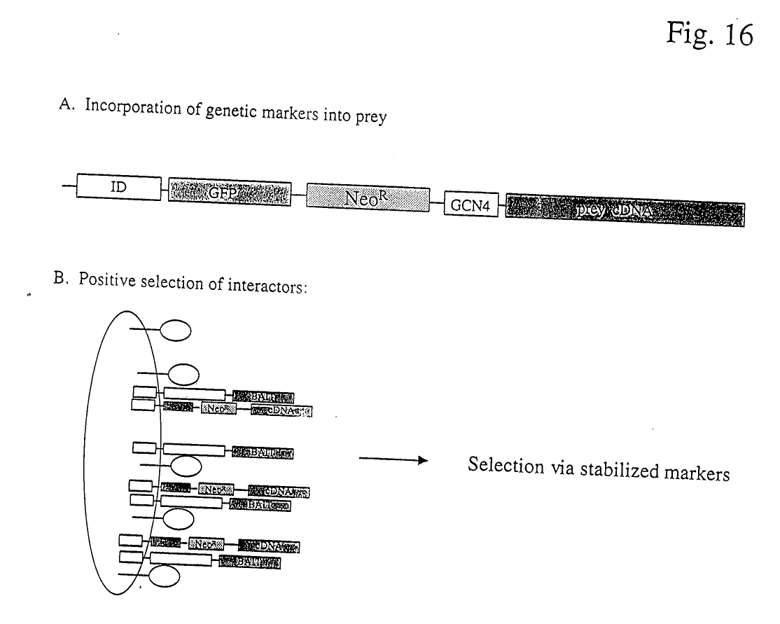

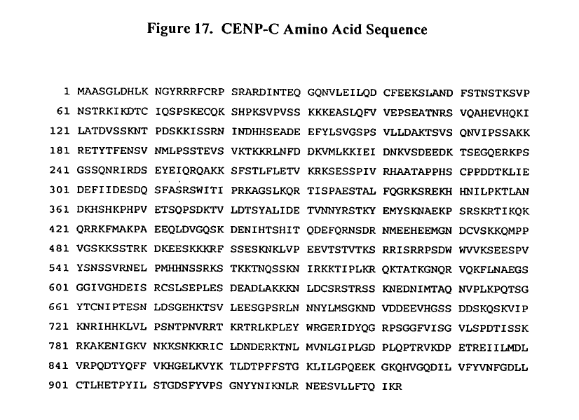

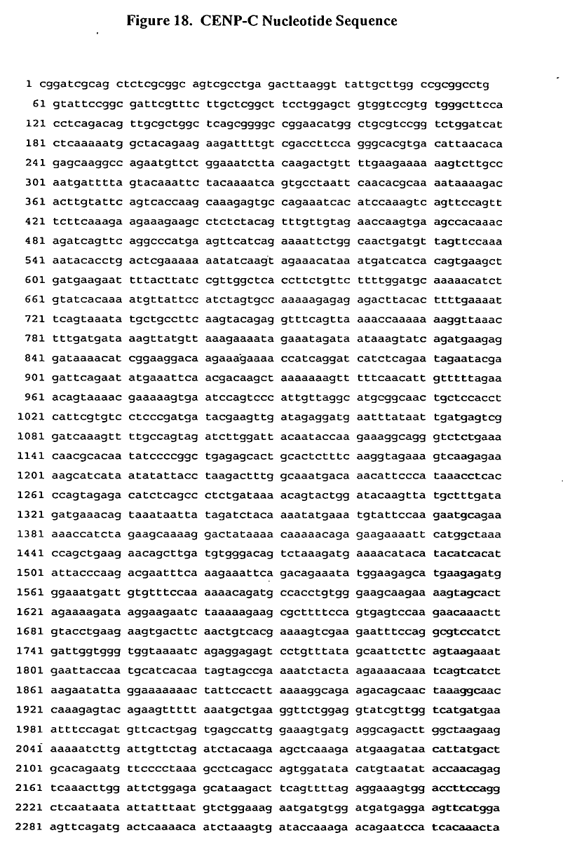

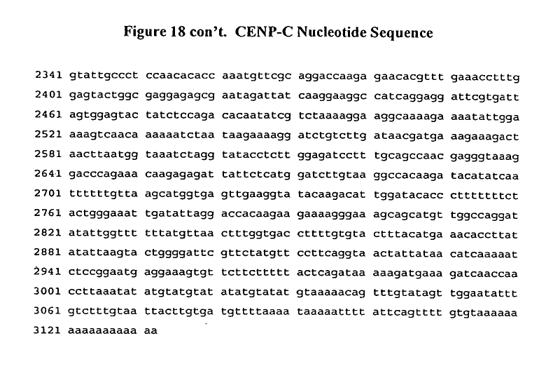

Another innovation that should enhance the system is to incorporate selectable markers into the prey molecule. These selectable markers could be neomycin resistance, hygromycin resistance, puromycin resistance, as well as others. Thus if the instability domain degrades non-targeted prey, the resistance marker will also be lost rendering the cell sensitive to the respective drugs (FIG. 16).

FIG. 16 is a schematic of the incorporation of selective genetic markers into the prey construct to aid in the detection of protein-protein interactions. The schematic shows the in-frame inclusion of a selectable gene marker into the prey construct. The docking of the prey molecule at the kinetochore will stabilize the marker allowing cell survival in the presence of drug.

All of the above-cited references and publications are hereby incorporated by reference.

### EQUIVALENTS

The present invention provides among other things novel methods for determining a druggable region on a protein. While specific embodiments of the subject invention have been discussed, the above specification is illustrative and not restrictive. Many variations of the invention will become apparent to those skilled in the art upon review of this specification. The appended claim is not intended to claim all such embodiments and variations, and the full scope of the invention should be determined by reference to the claim, along with its full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

1. A method for detecting protein interactions in a host cell, comprising:
(ii) providing a host cell including:
(a) a first nucleic acid coding sequence encoding a bait fusion protein comprising a bait polypeptide sequence fused to a targeting domain that targets the bait fusion protein to a subcellular location within the host cell;
(b) a second nucleic acid coding sequence encoding a prey fusion protein comprising a prey polypeptide sequence fused to one or more detection domains that can be detected in a transcriptionally-independent manner in the host cell to determine if the prey fusion protein is associated in a complex with the bait fusion protein or not;
(ii) detecting the subcellular location of the prey protein within the host cell; wherein accumulation of the prey fusion at the same subcellular pattern as the bait fusion indicates that the prey fusion protein is associated in a complex with the bait fusion protein.

2. A kit for detecting protein interactions, comprising:
(iii) a first expression construct including a coding sequence for a targeting domain and a ligation site flanking an end of the targeting domain coding sequence for ligating a coding sequence of a bait polypeptide sequence in frame with said targeting domain coding sequence to produce a bait fusion protein, said first expression construct operably linked to a transcriptional regulatory element; and
(iv) a second expression construct including a coding sequence for a detection domain and a ligation site flanking an end of the detection domain coding sequence for ligating a coding sequence of a prey polypeptide sequence in frame with said detection domain coding sequence to produce a prey fusion protein, said second expression construct operably linked to a transcriptional regulatory element,
wherein, the targeting domain localizes the bait fusion protein to a subcellular location within a host cell, and the detection domain can be detected in a transcriptionally-independent manner in the host cell to determine if the prey fusion protein is associated in a complex with the bait fusion protein or not.

3. The method of claim 1 or kit of claim 2, wherein the targeting domain localizes the bait fusion protein to subcellular comparment or organelle selected from the group consisting of the nucleus, nucleoli, telomeres, kinetochores, nuclear envelope, chromosomes, chromatin, cytoplasm, endoplasmic reticulum, Golgi, centrosome, transgolgi network, cytoplasmic vesicles, mitochondria, secretory vesicles, lysosome, plasma membrane, intracellular membrane vesicles, nuclear membranes, synapses and basolaternal membranes.

4. The method of claim 1 or kit of claim 2, wherein the targeting domain localizes the bait fusion protein to a kinetochore structure.

5. The method or kit of claim 4, wherein the targeting domain includes an amino acid sequence form a protein selected from the group consisting of CENP-A, CENP-B, CENP-C, CENP-E, CENP-F, Bub1, Bub3, MAD3L and MAD2, or a homologous sequence thereto localizes the bait fusion protein to a kinetochore structure.

6. The method or kit of claim 5, wherein the targeting domain comprises at least amino acids 373-943 of the human CENP-C sequence or a sequence at least 80 percent identical thereto.

7. The method or kit of claim 4, wherein the targeting domain associates with the kinetochore structure with a dissociation constant (k_{d}) of 1 mM or less.

8. The method of claim 1 or kit of claim 2, wherein the targeting domain localizes the bait fusion protein to the nuclear envelope.

9. The method or kit of claim 8, wherein the targeting domain comprises an amino acid sequence from a protein selected from the group consisting of lamin A, lamin B, lamin C, emerins and porins, or a portion thereof capable of targeting the bait fusion protein to the nuclear envelope.

10. The method of claim 1 or kit of claim 2, wherein the detection domain is a fluorescent polypeptide sequence or a luminescent polypeptide sequence.

11. The method or kit of claim 10, wherein the detection domain is all or a fluorescent portion of a green fluorescent protein sequence.

12. The method of claim 1 or kit of claim 2, wherein localization of the prey fusion proteins can be determined within 120 minutes of expression.

13. The method of claim 1 or kit of claim 2, wherein the prey fusion protein further includes an instability sequence which renders the prey fusion protein with a shorter intracellular half-life when not associated in complexes with the bait fusion protein relative to when it is.

14. The method or kit of claim 13, wherein the instability sequence comprises at least amino acids 249-323 of the human CENP-C sequence or a homologous sequence thereto.

15. The method of claim 1 or kit of claim 2, wherein either or both of the bait and prey fusion proteins includes a rescue sequence.

16. The method or kit of claim 15, wherein the rescue sequence is selected from the group consisting of His₆ tag, myc tag, flu tag, lacZ, GST, Strep tag I and Step tag II.

17. The method of claim 1 or kit of claim 2, wherein the bait and/or prey fusion protein includes an oligomerization domain.

18. The method of claim 1 or kit of claim 2, wherein the coding sequences for said bait and prey fusion proteins are operably linked to the same transcriptional regulatory sequence.

19. The method of claim 1 or kit of claim 2, wherein the coding sequences for said bait and prey fusion proteins are operably linked to different transcriptional regulatory sequences.

20. The method of claim 1 or kit of claim 2, wherein the coding sequences for said bait and prey fusion proteins are provided on the same expression vector.

21. The method of claim 1 or kit of claim 2, wherein the coding sequences for said bait and prey fusion proteins are provided on different expression vectors.

22. The method of claim 1 or kit of claim 2, wherein at least one of the coding sequences are provided as part of an integrative vector.

23. The method of kit of claim 22, wherein the vector is a retroviral vector.

24. The method of claim 1 or kit of claim 2, wherein at least one of the coding sequences are provided as part of an episomal vector.

25. The method of claim 1 or kit of claim 2, wherein at least one of the coding sequences are provided as part of a vector which includes a recovery element.

26. The method of claim 1 or kit of claim 2, wherein the host cell is a mammalian cell.

27. The method or kit of claim 26, wherein the host cell is a human cell.

28. The method of claim 1, wherein the subcellular location of the prey protein is determined in the presence of a test agent contacted with the cell.

29. The method of claim 28, wherein the test agent is a small organic molecule.

30. The method of claim 28, carried out consecutively or simultaneously for a library of at least 100 different test agents.

31. The method of claim 28, wherein the test agent includes a portion which is predetermined to bind to one the bait or prey fusion protein, and a test portion which being tested for binding to the other fusion protein.

32. The method of claim 28, wherein the ability of the test compound to inhibit association of the prey fusion protein in a complex with the bait fusion protein is determined.

33. The method of claim 28, wherein the ability of the test compound to potentiate association of the prey fusion protein in a complex with the bait fusion protein is determined.

34. The method of claim 30, wherein the identity of test agents in the library which inhibit or potentiate association of the prey fusion protein in a complex with the bait fusion protein is determined.

35. The method of claim 31, carried out for a library of at least 100 different test agents having varied test portions amongst members of the library.

36. The method of claim 28, comprising the further step of formulating a pharmaceutical preparation including one or more compounds identified as inhibitors or potentiators of the association of the prey fusion protein in a complex with the bait fusion protein, or analogs thereof.

37. The method of claim 1, wherein the subcellular location of the prey protein is determined after induction of the host cell with an agent the causes post-translational modification of proteins in the host cell.

38. The method of claim 1, wherein the subcellular location of the prey protein is determined using flow cytometry analysis.

39. The method of claim 1, wherein the subcellular location of the prey protein is determined using microscopy.

40. A method for detecting protein interactions in a host cell, comprising:
(i) providing a host cell culture, the cells of which include:
(c) a first nucleic acid coding sequence encoding a bait fusion protein comprising a bait polypeptide sequence fused to a targeting domain that targets the bait fusion protein to a subcellular location within the host cell, and
(d) a second nucleic acid coding sequence encoding a prey fusion protein comprising a prey polypeptide sequence fused to one or more detection domains that can be detected in a transcriptionally-independent manner in the host cell to determine if the prey fusion protein is associated in a complex with the bait fusion protein or not,
wherein the culture is a variegated mixture of cells containing different prey polypeptide sequences and/or different bait polypeptide sequences;
(ii) selecting cells from the culture in which the prey fusion protein is localized in the cell in the same subcellular pattern as the bait fusion protein;
(iii) identifying the sequence of the bait and prey fusion proteins from the selected cells

41. The method of claim 40, wherein the culture includes at least 100 different bait and/or prey polypeptide sequences.

42. The method of claim 40, wherein only one of the bait or prey polypeptide sequences is variegated in the culture.

43. A method for conducting a drug discovery business, comprising:
(vi) by the method of claim 1, identifying a protein complex for which an agent that inhibits or potentiates the formation or activity of the complex is desired;
(vii) generating a drug screening assay for identifying agents that inhibit or potentiate the formation or activity of the complex;
(viii) conducting animal toxicity profiles on a agent identified in step (ii), or an analog thereto;
(ix) manufacturing a pharmaceutical preparation of an agent having a suitable animal toxicity profile; and
(x) marketing the pharmaceutical preparation to healthcare providers.

44. A method for conducting a drug discovery business, comprising:
(vi) by the method of claim 1, identifying a protein complex which is mediated by post-translational modification and for which an agent that inhibits or potentiates the post-translational modification is desired;
(vii) generating a drug screening assay for identifying agents that inhibit or potentiate the post-translational modification and effect the formation of the protein complex;
(viii) conducting animal toxicity profiles on a agent identified in step (ii), or an analog thereto;
(ix) manufacturing a pharmaceutical preparation of an agent having a suitable animal toxicity profile; and
(x) marketing the pharmaceutical preparation to healthcare providers.

45. A method for conducting a bioinformatics business, comprising:
(iii) by the method of claim 1, identifying networks of protein complexes;
(iv) generating a database including information identifying interactions of different proteins in a signal pathway and information identifying the proteins.

46. A system for analyzing protein complexes in cells, comprising a flow cytometer for analyzing cells and determining if a fluorescent signal is dispersed in a cell or localized to kinetochore structures.

47. The system of claim 46, including a microprocessor for comparing the flow spectra of cells and distinguishing between a diffuse pattern of fluorescence in the cells and a kinetochore-localized pattern.

48. A system for analyzing protein complexes in cells, comprising a microscope having a camera mounted therein for analyzing cells in a field of vision of the microscope, and a microprocessor for processing images obtained from said camera and determining if a fluorescent signal is dispersed in a cell or localized to kinetochore structures.

49. The system of claim 48, further comprising an cell picking robot which is controlled by said microprocessor and isolates cells which the microprocessor has determined have a fluorescent signal localized to kinetochore structures.
